(19) United States
(12) Patent Application Publication
(10) Pub. No.: US 2011/0281300 A1
(43) Pub. Date: Nov. 17, 2011

(54) PREPARATION METHOD OF RECOMBINANT PROTEIN BY USE OF A FUSION EXPRESSION PARTNER

(75) Inventors: Jeewon Lee, Seoul (KR); Kyung-Yeon Han, Seoul (KR); Jong-Am Song, Seoul (KR); Jin-Seung Park, Seoul (KR); Hyuk-Seong Seo, Seoul (KR); Keum-Young Ahn, Gyeonggi-do (KR); Eun-Jung Lee, Gyeonggi-do (KR); Jong-Hwan Lee, Seoul (KR); Soo-Jung Kwon, Seoul (KR)

(73) Assignee: Nutrex Technology Co., Ltd, Seoul (KR)

(21) Appl. No.: 13/108,518

(22) Filed: May 16, 2011

Related U.S. Application Data
(62) Division of application No. 12/676,982, filed on Mar. 8, 2010, now abandoned.

(30) Foreign Application Priority Data
Sep. 6, 2007 (KR) 10-2007-0090366
Sep. 6, 2007 (KR) 10-2007-0090367
Sep. 6, 2007 (KR) 10-2007-0090368
Sep. 6, 2007 (KR) 10-2007-0090370
Sep. 6, 2007 (KR) 10-2007-0090371

(51) Int. Cl. C12P 21/00; C12N 15/63; C12N 1/21; C12N 9/00

(52) U.S. Cl. 435/69.3; 435/69.1; 435/69.6; 435/183; 435/320.1; 435/252.33

(57) ABSTRACT

The present invention relates to a preparation method using a fusion expression partner. The method includes preparing a polynucleotide encoding a fusion expression partner selected from the group consisting of SlyD (FKBR-type peptidyl prolyl cis-trans isomerase), Crr (glucose-specific phosphotransferase (PTS) enzyme IIA component), RpoS (RNA polymerase sigma factor), PotD (Spermidine/putrescine-binding periplasmic protein), and RpoA (RNA polymerase alpha subunit), and an expression vector linking a polyDNA fragment of a heterologous protein, preparing a transformant by introducing the expression vector into a host cell, inducing the expression of a recombinant protein by culturing a transformant, and obtaining the expression. In the preparation method of the recombinant protein, the heterologous protein may enhance the water-solubility and folding of the recombinant protein, overcome the limitations about the water-solubility and folding which the conventional fusion expression partners have, and be used widely in the production of pharmaceutical and industrial proteins.

### TECHNICAL FIELD

The present invention relates to a preparation method using a fusion expression partner selected from the group consisting of SlyD (FKBR-type peptidyl prolyl cis-trans isomerase), Crr (glucose-specific phosphotransferase enzyme IIA component), RpoS (RNA polymerase sigma factor), PotD (Spermidine/putrescine-binding periplasmic protein), and RpoA (RNA polymerase alpha subunit).

### BACKGROUND ART

The proteins produced by the current bio-engineering technology may be generally represented by proteins for medical and research purposes, such as immune regulatory and enzyme inhibitors and hormones, and industrial proteins, such as proteins for diagnosis and reaction addition enzymes, and the development of production process technologies and their industrialization are being focused on these two proteins. Particularly, when a useful recombinant protein is produced using a recombinant microorganisms technology, an E. coli which has advantages, that it has well known genetic information and various vector systems, and can be cultured fast in relatively low-priced medium at high concentration is being widely used for research and commercial purposes.

In the production of recombinant proteins from E. coli, various expression vectors with a powerful inducible promoter have been developed and used for production of heterologous proteins. However, in the case of an E. coli as a host cell, the protein to be prepared is hydrolized by proteases and its yield tends to be low. It is known that this tendency is particularly obvious in the expression of polypeptides with a 10 kDa or less. Furthermore, when recombinant proteins are overexpressed in E. coli, it is known that an insoluble aggregate known as an inclusion body is often formed. In the case of a polypeptide expressed as an inclusion body, it has a disadvantage that the purity of the target polypeptide in the inclusion body can be reduced because a folding intermediate is randomly bonded to other protein impurities, including chaperons, ribosomes, and initial factors, through intermolecular disulfide bonds or hydrophobic interactions. To convert these expressed proteins into active forms, the proteins must be dissolved using denaturants, such as guanidine hydrochloride, urea, etc., and refolded through dilution. During the refolding process, production yield can be decreased, such that the proteins fail to be refolded as active forms (Marston F A, Biochem. J. 240(1): 1-12, 1986).

To obtain a high yield of recombinant proteins as active forms in E. coli, various methods have been attempted, including a low-temperature expression performance by decreasing the expression rate of proteins in E. coli and increasing the acceptability of heterologous proteins (Hammarstrom et al., Protein Sci. 11:313-321, 2002), the use of various promoters or optimization of induction conditions (Qing et al., Nat. Biotechnol. 22:877-882, 2004), and a simultaneous expression of molecular chaperones or protein folding regulators and heterologous proteins (de Marco & De Marco, J Biotechnol. 109:45-52, 2004). However, expression method by fusing a heterologous protein with a fusion expression partner which aids in purification and folding of the heterologous protein is the most common.

In fact, various fusion expression partners which induce a high expression of heterologous proteins within E. coli have been studied and reported (Esposito & Chatterjee, Curr Opin Biotechnol. 17:353-358 2006; Kapust & Waugh, Protein Sci. 8:1668-1674, 1999; Sachdev & Chirgwin, Biochem. Biophys. Res. Commun. 244:933-937, 1998). Although a number of fusion expression partners have been developed and used, representative fusion expression partners which have been studied the most include maltose binding protein (MBP), glutathione-S-transferase (GST), thioredoxin (Trx), NusA, and the like. In case of maltose binding protein, wide hydrophobic voids resulting from the aggregation of hydrophobic amino acids effectively shield hydrophobic site's of newly-synthesized proteins and prevent heterologous proteins from becoming insoluble inclusion bodies, and thioredoxins help the disulfide bondage of heterologous proteins. NusA, when overexpressed in E. coli, shows an excellent ability to be folded as an active form and induces correct foldings of heterologous proteins subsequently expressed (Bach H et al., J Mol Biol 312:79-93, 2001; Edward R L et al., Nat Biotechnol 11:187-193, 1993; Davis G D et al., Biotechnol Bioeng 65:382-388, 1999). It is known that these fusion expression partners have an advantage that the fusion expressed heterologous proteins using an affinity chromatography method may be easily purified, and help the folding of the heterologous proteins utilizing each of other molecular biological properties. However, because these fusion expression partners are relatively big compared to heterologous proteins, they have disadvantages that the yield of heterologous proteins are considerably decreased with the size of the fusion part and the partners are not universally applicable to either proteins for medical purposes or industrially useful proteins. In addition, although heterologous proteins may be induced as water-soluble forms, it is hard for them to be induced into the expression of active forms which can carry out their inherent functions, and there is a irrationality in processes that a process of removing fusion expression partners should be added for them to be used for suitable purposes.

Hitherto, the domestic recombinant proteins produced industrially by the bioengineering technology have been focused on the development of production processes, by which recombinant proteins may be expressed. The development of expression systems, which are a core source technology, is under way as a supplementary technology which is at a copy or an improving level. Secretary and membrane proteins, which are very important for commercial or pharmaceutical reasons, are difficult-to-express proteins to form insoluble inclusion bodies in E. coli when they are expressed in E. coli Thus, the development is retarded. To overcome these, it is important to secure a source technology on the expression system of difficult-to-express proteins in E. coli.

As described above, each fusion expression partner has different molecular biological properties and different mechanisms in helping the folding of the fused recombinant proteins. Thus, the same effects are not shown in all the proteins. To construct an expression system using a fusion expression partner which helps the folding of recombinant proteins more universally, a new concept of a fusion expression partner library should be constructed by discovering a fusion expression partner from different points of view which deviate from the properties of conventional fusion expression partners.

It is known in the previous report (Hoffman F & Rinas U, Adv Biochem Eng Biotechnol, 89:73-92, 2004) that the overexpression of recombinant proteins in E. coli imparts similar effects, for example, stress (thermal shock, amino acid depletion, etc).

### DISCLOSURE

### Technical Problem

The object of the present invention is to provide a preparation method of recombinant proteins using a universal fusion expression partner available in various heterologous proteins as well as separating active proteins without using denaturants or reducing agents, in production of heterologous proteins from transgenic microorganisms as an insoluble inclusion body.

### Technical Solution

The present inventors have attempted to develop a new conceptual fusion expression partner. After imparting stress which inhibits a correct folding to the E. coli and producing various heterologous proteins using overexpressed proteins as fusion expression partners through a gene recombinant technology, we confirmed that the amount of water-soluble expressions was increased with regard to various heterologous proteins and the production of recombinant proteins which are unchanged in enzyme activity was possible, and completed the present invention.

The present invention provides a preparation method of recombinant proteins using a universal fusion expression partner.

The present invention also provides an expression vector for production of recombinant proteins, including a polynucleotide encoding a universal fusion expression partner and a polynucleotide encoding a heterologous protein.

In addition, the present invention provides transformants transduced by the expression vectors.

Furthermore, the present invention provides recombinant fusion expression proteins prepared by the method of recombinant proteins.

Hereinafter, the terms as used in the present invention will be described.

“Heterologous protein or target protein” means a protein which those skilled in the art are attempting to produce in a large amount, and may be expressed in transformants by inserting a polynucleotide encoding the protein in a recombinant expression vector.

“Recombinant protein or fusion protein” means a protein, in which another protein is linked with or another amino acid sequence is added to the N-terminal or C-terminal of the sequence of the original heterologous protein.

“Expression vector” refers to a linear or round DNA molecule consisting of fragments encoding polypeptides of interest, operably linked with an additional fragment provided in the transcription of the expression vector. That additional fragment includes a promoter and a termination sequence. The expression vector includes one or more origins of replication, one or more selection markers, a polyadenylation signal, and the like. The expression vector is generally induced from a plasmid or virus DNA, or contains both the elements.

Hereinafter, the present invention will be described in more detail.

1. The present invention provides a preparation method of recombinant proteins, including the following:

1) preparing an expression vector which links a polynucleotide encoding a fusion expression partner selected from the group consisting of SlyD (FKBP-type peptidyl-prolyl cis-tans isomerase), Crr [glucose-specific phosphotransferase (PTS) enzyme IIA component], Rpos (RNA polymerase sigma factor), PotD (Spermidine/putrescine-binding periplasmic protein), and RpoA (RNA polymerase alpha subunit) with a poly DNA fragment encoding a heterologous protein;

2) preparing a transformant by introducing the expression vector into a host cell; and

3) inducing and obtaining an expression of a recombinant protein by culturing the transformant.

In the method, it is desirable that the SlyD, Crr, RpoS, PotD, and RpoA are represented by SEQ ID NO: 1, 4, 7, 10, and 13, respectively.

The heterologous protein according to the step 1) desirably has a biological activity of a protein selected from the group consisting of, but not limited to an antigen, an antibody, a cell receptor, an enzyme, a structural protein, a serum, and a cell protein. Furthermore, all the proteins that those skilled in the art desire are possible, and various heterologous proteins with a biological activity of a protein selected from the group consisting of particularly, a medical, research, and industrial protein, for example, an antigen, an antibody, a cell receptor, an enzyme, a structural protein, a serum, and a cell protein may be expressed as recombinant proteins.

1) SlyD

It is known that a trigger factor (hereinafter, referred to as ‘Tig’), one of the molecular chaperones of E. coli, binds to a ribosome with a polypeptide when the protein is expressed and provides a space to protect hydrophobic amino acids from the outside before a perfect 3D-structure is formed by a polypeptide during synthesis, in order not to form an insoluble inclusion body through a non-specific interaction (Ferbitz et al., Nature 431:590-596, 2004). SlyD is a protein with a function of a peptidyl-proplyl isomerase (PPIase), corresponding to a middle domain of Tig (Hotternrott et al., J Biol Chem 272:15697-15701, 1997). Like the Tig, it is expected that during a formation of a heterologous protein fused to the 3′-terminal, it forms a protective space between the heterologous protein and the ribosome and controls a formation of an insoluble inclusion body of the polypeptide prior to the 3D-structure formation. NusA, one of the well known fusion proteins, is selected as a fusion expression partner for expression of the heterologous protein through a prediction program that a highly water-soluble protein may be produced when an overexpression is induced in E. coli (Davis et al., Biotechnol Bioeng 65:382-388, 1999). The SlyD exhibited 98% solubility when applied to the Davis program, and the high folding ability indicates the possibility of the SlyD as a fusion protein.

There are many cases that heterologous proteins are expressed as insoluble inclusion bodies in E. coli Accordingly, the present invention attempted to enhance the water-soluble expression of recombinant proteins, using an SlyD whose expression was increased when stresses inhibiting the correct folding of proteins were applied (See FIG. 1), as a fusion expression partner. Even under conditions inhibiting the folding of proteins, the activity of the protein is maintained and the amount of expression is increased. Because the protein exhibits stability in the protein structure and excellence in the folding ability as well as the possibility of being involved in bio-mechanisms of E. coli, it may be effectively used as a fusion protein.

Specifically, when stresses inhibiting the correct folding of proteins were applied such as culturing E. coli at 48° C., higher than 37° C., its optimum growth temperature, E. coli proteins showing an increase in expression were screened. When the protein was analyzed and identified, it was confirmed that the amount of expression of the E. coli SlyD (FKBP-type peptidyl-prolyl cis-trans isomerase SlyD) was increased (See Table 1). Generally, when growth conditions deviate from the optimum conditions, a reaction mechanism for adapting to changed environmental conditions occurs. Simultaneously, structurally-unstable proteins form insoluble inclusion bodies. The growth of E. coli may be maintained, but after the E. coli was cultured at temperatures higher than the optimum culture temperature, it was confirmed that a 2D-electrophoresis analysis for comparison of changes in E. coli proteins was followed by 3.37 times increase in the expression amount of a certain protein (See FIG. 2). The analysis and identification results of the protein with its expression amount increased, using a Matrix-Assisted Laser Desorption/Ionization-Time of Flight Mass Spectrometer (MALDI-TOF-MS), confirmed that it was SlyD (See Table 1).

In the present invention, SlyD was fusion expressed with 10 other proteins, known to form insoluble inclusion bodies when expressed separately without a fusion expression partner in E. coli in order to verify the possibility of the SlyD as a fusion expression partner. As a representative medical and research protein, human minipro-insulin (hereinafter, referred to as “mp-INS”), human epidermal growth factor (hereinafter, referred to as “EGF”), human prepro-ghrelin (hereinafter, referred to as “ppGRN”), human interleukin-2 (hereinafter, referred to as “hIL-2”), human activation induced cytidine deaminase, referred to as “AID”), human glutamate decarboxylase (hereinafter, referred to as “GAD₄₄₆₋₅₈₅”), cutinase (hereinafter, referred to as “CUT”) derived from Pseudomonas putida, human ferritin light chain (hereinafter, referred to as “hFTN-L), human granulocyte colony-stimulating factor (hereinafter, referred to as “G-CSF”), and cold autoinflammatory syndromel (NALP3) Nacht domain (hereinafter, referred to as “Nacht”) were fusion-expressed. As an industrial protein, glutamate decarboxylase₄₄₈₋₅₈₅ (hereinafter, referred to as “GAD₄₄₈₋₅₈₅”) known as a prognostic marker in type 1 diabetes mellitus, and cutinase (hereinafter, referred to as “CUT”), derived from Pseudomonas putida, which is used in the pretreatment of fiber and emerging as an environmentally-friendly enzyme for its degradability of plastics were selected as a heterologous protein. After each of the expression vectors inserted into the carboxyl terminal of the SlyD was prepared (See FIG. 7), it was transformed in the E. coli and produced therefrom in the form of a recombinant protein. When the amount of expression of each recombinant protein was measured, it was confirmed that the expression of the heterologous protein fused with the SlyD was more abundant in water-soluble expression than the expressions of most insoluble inclusion bodies (See FIG. 12). It was also confirmed that the amount of water-soluble expression of the heterologous protein fused with the SlyD is increased significantly compared to that of water-soluble expression of a singly expressed heterologous protein (See FIG. 13).

Furthermore, the presence of a PNP specific proteolytic activity of a recombinant cutinase (SlyD::CUT), an industrial protein which can be used without any removal of a fusion expression partner to verify the activity of the recombinant protein by the method, was confirmed, leading to the completion of the present invention (See FIG. 19).

2) Crr

The present invention attempted to enhance the water-soluble expression of recombinant proteins, using a Crr whose expression was increased when stresses inhibiting the correct folding of proteins were applied, as a fusion expression partner.

Specifically, when stresses inhibiting the correct folding of proteins were applied by the addition of the GdnHCl, exhibiting a proteolytic effect, E. coli proteins showing an increase in expression were screened (See FIG. 3). When the protein was analyzed and identified, it was confirmed that the amount of expression of the E. coli Crr [glucose-specific phosphotransferase (PTS) enzyme IIA component] was increased (See Table 2). When a protein with the water-soluble expression against the proteolytic stress increased, is used as a fusion expression partner, the high level of the water-soluble expression of the heterologous protein may be maintained. After GdnHCl was added to the medium at a concentration sufficient for the E. coli to keep its growth, it was confirmed that a 2D-electrophoresis analysis for comparison of changes in E. coli proteins was followed by 2.2 times increase in the amount of the expression of a certain protein (See FIG. 3). The analysis and identification results of the protein with its expression amount increased, using a MALDI-TOF-MS, confirmed that it was Crr (See Table 1).

In the present invention, Crr was fusion expressed with 10 other proteins, known to form insoluble inclusion bodies when expressed separately without a fusion expression partner in E. coli in order to verify the possibility of the Crr as a fusion expression partner. As a representative medical and research protein, mp-INS, EGF, ppGRN, hIL-2, AID, GAD₄₄₈₋₅₈₅, CUT, hFTN-L, G-CSF, and Nacht were fusion-expressed. As an industrial protein, GAD₄₄₈₋₅₈₅ known as a prognostic marker in type 1 diabetes mellitus, and CUT derived from Pseudomonas putida which is used in the pretreatment of fiber and emerging as an environmentally-friendly enzyme for its degradability of plastics were selected as a heterologous protein. After each of the expression vectors inserted into the carboxyl terminal of the Crr was prepared (See FIG. 8), it was transformed in the E. coli and produced therefrom in the form of a recombinant protein. When the amount of expression of each recombinant protein was measured, it was confirmed that the expression of the heterologous protein fused with the Crr was more abundant in water-soluble expression than the expressions of most insoluble inclusion bodies (See FIG. 15). It was also confirmed that the amount of water-soluble expression of the heterologous protein fused with the Crr is increased significantly compared to that of water-soluble expression of a singly expressed heterologous protein (See FIG. 13).

Furthermore, the presence of a PNP specific proteolytic activity of a recombinant cutinase (Crr::CUT), an industrial protein which can be used without any removal of a fusion expression partner to verify the activity of the recombinant protein by the method, was confirmed (See FIG. 16).

3) RpoS

The present invention attempted to enhance the water-soluble expression of recombinant proteins, using a Rpos whose expression was increased when stresses inhibiting the correct folding of proteins were applied, as a fusion expression partner.

Specifically, when stresses inhibiting the correct folding of proteins were applied by the addition of the GdnHCl, exhibiting a proteolytic effect, E. coli proteins showing an increase in expression were screened (See FIG. 4). When the protein was analyzed and identified, it was confirmed that the amount of expression of the E. coli RpoS (RNA polymerase sigma factor) was increased (See Table 3). When a protein with the water-soluble expression against the proteolytic stress increased is used as a fusion expression partner, the high level of the water-soluble expression of the heterologous protein may be maintained. After GdnHCl was added to the medium at a concentration sufficient for the E. coli to keep its growth, it was confirmed that a 2D-electrophoresis analysis for comparison of changes in E. coli proteins was followed by about 6 times increase in the amount of the expression of a certain protein (See FIG. 4). The analysis and identification results of the protein with its expression amount increased, using a MALDI-TOF-MS, confirmed that it was RpoS (See Table 3).

In the present invention, RpoS was fusion expressed with 10 other proteins, known to form insoluble inclusion bodies when expressed separately without a fusion expression partner in E. coli in order to verify the possibility of the RpoS as a fusion expression partner. As a representative medical and research protein, mp-INS, EGF, ppGRN, hIL-2, AID, GAD₄₄₈₋₅₈₅, CUT, hFTN-L, G-CSF, and Nacht were fusion-expressed. As an industrial protein, GAD₄₄₈₋₅₈₅ known as a prognostic marker in type 1 diabetes mellitus, and CUT derived from Pseudomonas putida which is used in the pretreatment of fiber and emerging as an environmentally-friendly enzyme for its degradability of plastics were selected as a heterologous protein. After each of the expression vectors inserted into the carboxyl terminal of the Rpos was prepared (See FIG. 8), it was transformed in the E. coli and produced therefrom in the form of a recombinant protein. When the amount of expression of each recombinant protein was measured, it was confirmed that the expression of the heterologous protein fused with the Rpos was more abundant in water-soluble expression than the expressions of most insoluble inclusion bodies (See FIG. 18). It was also confirmed that the amount of water-soluble expression of the heterologous protein fused with the RpoS is increased significantly compared to that of water-soluble expression of a singly expressed heterologous protein (See FIG. 19).

Furthermore, the presence of a PNP specific proteolytic activity of a recombinant cutinase (RpoS::CUT), an industrial protein which can be used without any removal of a fusion expression partner to verify the activity of the recombinant protein by the method, was confirmed (See FIG. 16).

4) PotD

The present invention attempted to enhance the water-soluble expression of recombinant proteins, using a PotD whose expression was increased when stresses inhibiting the correct folding of proteins were applied, as a fusion expression partner.

Specifically, when stresses inhibiting the correct folding of proteins were applied by the addition of the GdnHCl, exhibiting a proteolytic effect, E. coli proteins showing an increase in expression were screened (See FIG. 5). When the protein was analyzed and identified, it was confirmed that the amount of expression of the E. coli PotD (Spermidine/putrescine-binding periplasmic protein) was increased (See Table 4). When a protein with the water-soluble expression against the proteolytic stress increased is used as a fusion expression partner, the high level of the water-soluble expression of the heterologous protein may be maintained. After GdnHCl was added to the medium at a concentration sufficient for the E. coli to keep its growth, it was confirmed that a 2D-electrophoresis analysis for comparison of changes in E. coli proteins was followed by about 3.5 times increase in the amount of the expression of a certain protein (See FIG. 5). The analysis and identification results of the protein with its expression amount increased, using a MALDI-TOF-MS, confirmed that it was PotD (See Table 4).

In the present invention, PotD was fusion expressed with 10 other proteins, known to form insoluble inclusion bodies when expressed separately without a fusion expression partner in E. coli in order to verify the possibility of the PotD as a fusion expression partner. As a representative medical and research protein, mp-INS, EGF, ppGRN, hIL-2, AID, GAD₄₄₈₋₅₈₅, CUT, hFTN-L, G-CSF, and Nacht were fusion-expressed. As an industrial protein, GAD₄₄₈₋₅₈₅ known as a prognostic marker in type 1 diabetes mellitus, and CUT derived from Pseudomonas putida which is used in the pretreatment of fiber and emerging as an environmentally-friendly enzyme for its degradability of plastics were selected as a heterologous protein. After each of the expression vectors inserted into the carboxyl terminal of the PotD was prepared (See FIG. 8), it was transformed in the E. coli and produced therefrom in the form of a recombinant protein. When the amount of expression of each recombinant protein was measured, it was confirmed that the expression of the heterologous protein fused with the PotD was more abundant in water-soluble expression than the expressions of most insoluble inclusion bodies (See FIG. 22). It was also confirmed that the amount of water-soluble expression of the heterologous protein fused with the PotD is increased significantly compared to that of water-soluble expression of a singly expressed heterologous protein (See FIG. 22).

Furthermore, the presence of a PNP specific proteolytic activity of a recombinant cutinase (PotD::CUT), an industrial protein which can be used without any removal of a fusion expression partner to verify the activity of the recombinant protein by the method, was confirmed (See FIG. 23).

5) RpoA

The present invention attempted to enhance the water-soluble expression of recombinant proteins, using a RNA polymerase α subunit whose expression was increased when stresses inhibiting the correct folding of proteins were applied, as a fusion expression partner.

Specifically, when stresses inhibiting the correct folding of proteins were applied by the addition of the 2-hydroxyethyldisulfide (2-HEDS), known as an oxidizing agent, E. coli proteins showing an increase in expression were screened (See FIG. 6). When the protein was analyzed and identified, it was confirmed that the amount of expression of the E. coli RNA polymerase α subunit (hereinafter, referred to as “RpoA”) was increased (See Table 5). When a protein with the water-soluble expression against the proteolytic stress increased is used as a fusion expression partner, the high level of the water-soluble expression of the heterologous protein may be maintained. After 2-HEDS was added to the medium at a concentration sufficient for the E. coli to keep its growth, it was confirmed that a 2D-electrophoresis analysis for comparison of changes in E. coli proteins was followed by about 1.5 times increase in the amount of the expression of a certain protein (See FIG. 5). The analysis and identification results of the protein with its expression amount increased, using a MALDI-TOF-MS, confirmed that it was RpoA (See Table 5).

In the present invention, RpoA was fusion expressed with 10 other proteins, known to form insoluble inclusion bodies when expressed separately without a fusion expression partner in E. coli in order to verify the possibility of the RpoA as a fusion expression partner. As a representative medical and research protein, mp-INS, EGF, ppGRN, hIL-2, AID, GAD₄₄₈₋₅₈₅, CUT, hFTN-L, G-CSF, and Nacht were fusion-expressed. As an industrial protein, GAD₄₄₈₋₅₈₅ known as a prognostic marker in type 1 diabetes mellitus, and CUT derived from Pseudomonas putida which is used in the pretreatment of fiber and emerging as an environmentally-friendly enzyme for its degradability of plastics were selected as a heterologous protein. After each of the expression vectors inserted into the carboxyl terminal of the PotD was prepared (See FIG. 11), it was transformed in the E. coli and produced therefrom in the form of a recombinant protein. When the amount of expression of each recombinant protein was measured, it was confirmed that the expression of the heterologous protein fused with the RpoA was more abundant in water-soluble expression than the expressions of most insoluble inclusion bodies (See FIG. 25). It was also confirmed that the amount of water-soluble expression of the heterologous protein fused with the RpoA is increased significantly compared to that of water-soluble expression of a singly expressed heterologous protein (See FIG. 26).

Furthermore, the presence of a PNP specific proteolytic activity of a recombinant cutinase (RpoA::CUT), an industrial protein which can be used without any removal of a fusion expression partner to verify the activity of the recombinant protein by the method, was confirmed (See FIG. 23).

2. The present invention provides an expression vector for production of recombinant proteins, including a polynucleotide encoding a fusion expression partner selected from the group consisting of SlyD, Crr, RpoS, PotD, and RpoA, and a polynucleotide encoding a heterologous protein.

It is desirable that the polynucleotide encoding the fusion expression partner linked with the polynucleotide encoding the heterologous protein is included in the expression vector.

In addition, a polynucleotide encoding a protein restriction enzyme recognition site is desirably linked between the polynucleotide encoding the fusion expression partner and the polynucleotide encoding the heterologous protein.

As a fusion expression partner, a polynucleotide, selected from the group consisting of SlyD, Crr, RpoS, PotD, and RpoA, linked with a polynucleotide encoding a heterologous protein is included in a backbone vector, and as a backbone vector, a variety of transformable vectors may be used in the E. coli selected from the group consisting of, but not limited to, pT7, pET/Rb, pGEX, pET28a, pET-22b(+), and pGEMX. The expression vector of the present invention may express the fusion expression partner and the heterologous protein as recombinant proteins by including the insertion sites of the genes encoding the fusion expression partner and the heterologous protein in a sequence.

Furthermore, a polynucleotide encoding a protein restriction enzyme recognition site may be linked between a polynucleotide operably encoding a fusion expression partner in the fusion expression partner gene and the heterologous protein of the present invention. When the heterologous protein to be fused is an industrial protein, the fusion expression partner may degrade the function of the heterologous protein, and the fusion expression partner of a medical protein is desirably removed because the protein may cause an antigen-antibody reaction. As for the protein restriction enzyme recognition site, one selected from the group consisting of Xa factor recognition site, enterokinase recognition site, genenase I recognition site, or furin recognition site may be used alone or in any combination of at least two of them.

In addition, a polynucleotide encoding a tag for separation and purification may be operably linked with the genes of the fusion expression partner or heterologous protein of the vector of the present invention, allowing the recombinant protein to be easily separated and purified. As for the tag for separation and purification, one selected from the group consisting of GST, poly-Arg, FLAG, poly-His, and c-myc may be used alone or in any combination of at least two of them. In one aspect of the present invention, the vector of the present invention may be expressed as a recombinant protein including the fusion expression partner, the heterologous protein, and the tag for separation and purification by incorporating the insertion sites of the fusion expression partner gene, the heterologous protein gene, and the tag for separation and purification into the pT7 backbone vector in series.

In one aspect of the present invention, the exogenous gene may be cloned through a restriction enzyme site, linked in frame with the polynucleotide when a polynucleotide encoding a protein restriction enzyme recognition site is used, and allowed to produce a heterologous protein in the original form when cut with a protein restriction enzyme after the secretion of the heterologous protein.

3. The present invention provides a transformant transduced with the expression vector.

The transformant may be obtained through transduction of the expression vector into a host cell known to those skilled in the art by a known method. Through this, a recombinant protein with both its water-solubility and folding improved may be obtained by those skilled in the art.

4. A recombinant fusion protein by a preparation method of the recombinant protein is provided.

A recombinant protein with both its water-solubility and folding improved may be obtained by those skilled in the art through a preparation method of the recombinant protein according to the present invention.

### DESCRIPTION OF DRAWINGS

FIG. 1 shows growth curves of the BL21 (DE3) culture at 37° C. (a control group) and at 49° C. (an experimental group), respectively.

FIG. 2 shows results analyzing the changes of the E. coli proteins under environmental stresses:

A: the results of 2-D PAGE (2-dimensional polyacrylamide gel electrophoresis); and

B: the increase in the amount of expression of the SlyD culture at 48° C.

FIG. 3 shows results analyzing the changes of the E. coli proteins under environmental stresses:

A: the results of 2-D PAGE; and

B: the increase in the amount of expression of the Crr by GdnHCl.

FIG. 4 shows results analyzing the changes of the E. coli proteins under environmental stresses:

A: the results of 2-D PAGE; and

B: the increase in the amount of expression of the RpoS by GdnHCl.

FIG. 5 shows results analyzing the changes of the E. coli proteins under environmental stresses:

A: the results of 2-D PAGE; and

B: the increase in the amount of expression of the PotD by GdnHCl.

FIG. 6 shows results analyzing the changes of the E. coli proteins under environmental stresses:

A: the results of 2-D PAGE; and

B: the increase in the amount of expression of the RpoA by 2-HEDS.

FIG. 7 shows the construction processes of a single expression vector of the heterologous protein and a fusion expression vector with a fusion expression partner.

FIG. 8 are cleavage maps of a single expression vector of the heterologous protein, a fusion expression vector with a fusion expression partner, and a fusion expression vector including a protein restriction enzyme recognition site:

A: a cleavage map of a single expression vector;

B: a cleavage map of a fusion expression vector; and

C: a cleavage map of a fusion expression vector including a protein restriction enzyme recognition site.

FIG. 9 are cleavage maps of a single expression vector of the heterologous protein, a fusion expression vector with a fusion expression partner, and a fusion expression vector including a protein restriction enzyme recognition site:

A: a cleavage map of a single expression vector;

B: a cleavage map of a fusion expression vector; and

C: a cleavage map of an expression vector for purification including 6 histidines.

FIG. 10 are cleavage maps of a single expression vector of the heterologous protein, a fusion expression vector with a fusion expression partner, and a fusion expression vector including a protein restriction enzyme recognition site:

A: a cleavage map of a single expression vector;

B: a cleavage map of a fusion expression vector; and

C: a cleavage map of a fusion expression including a protein restriction enzyme recognition site.

FIG. 11 shows the construction processes of a single expression vector of the heterologous protein and a fusion expression vector with a fusion expression partner.

FIG. 12 shows comparisons of the amounts of expression of recombinant proteins fusion-expressed with the SlyD in a water-soluble supernatant (S) and an insoluble inclusion body (IS).

FIG. 13 shows graphs comparing the amounts of water-soluble expression between a single expression recombinant protein and a recombinant expression fusion-expressed with the SlyD.

FIG. 14 shows the results measuring the resolutions of PNB () and PNP (◯) between a crude water-soluble protein of E. coli, which does not express the cutinase and a crude water-soluble protein of E. coli, which expresses a recombinant protein fusion-expressed with the SlyD in the form of SlyD::CUT.

FIG. 15 shows the results of the amount of a single expression of the target protein and comparison of the amounts of a recombinant protein fusion-expressed with the Crr in a water-soluble supernatant (S) and an insoluble inclusion body (IS).

FIG. 16 shows the results measuring the resolutions of PNB () and PNP (◯) between a crude water-soluble protein of E. coli, which does not express the cutinase and a crude water-soluble protein of E. coli, which expresses a recombinant protein fusion-expressed with the Crr in the form of Crr::CUT.

FIG. 17 indicates that a recombinant protein with its fusion expression partner removed keeps the water-soluble properties:

M: protein marker;

1: Crr::G-CSF water-soluble supernatant;

2: Crr::G-CSF insoluble inclusion body;

3: G-CSF water-soluble supernatant with the Crr removed using enterokinase; and

4: G-CSF insoluble inclusion body with the Crr removed using enterokinase.

FIG. 18 shows comparison of the amounts of water-soluble expression of a recombinant protein fusion-expressed with the RpoS in a water-soluble supernatant (S) and an insoluble inclusion body (IS).

FIG. 19 shows a comparison of the amounts of water-soluble expression between a single expressed recombinant protein and a recombinant fusion-expressed with the RpoS.

FIG. 20 shows the results measuring the resolutions of PNB () and PNP (◯) between a crude water-soluble protein of E. coli, which does not express the cutinase and a crude water-soluble protein of E. coli, which expresses a recombinant protein fusion-expressed with the RpoS in the form of RpoS::CUT.

FIG. 21 shows the results of purifying a recombinant protein using His tag:

A: purification results by the SDS-PAGE method;
M: protein marker;
1: crude water-soluble supernatant including RpoS::CUT-His₆;
2 & 3: purified RpoS::CUT-His₆;

B: purification results by HPLC

FIG. 22 shows the results of the amount of a single expression of the target protein and comparison of the amounts of a recombinant protein fusion-expressed with the Crr in a water-soluble supernatant (S) and an insoluble inclusion body (IS).

FIG. 23 shows the results measuring the resolutions of PNB () and PNP (◯) between a crude water-soluble protein of E. coli, which does not express the cutinase and a crude water-soluble protein of E. coli, which expresses a recombinant protein fusion-expressed with the RpoS in the form of PotD::CUT.

FIG. 24 indicates that a recombinant protein with its fusion expression partner removed keeps the water-soluble properties:

M: protein marker;

1: PotD::G-CSF water-soluble supernatant;

2: PotD::G-CSF insoluble inclusion body;

3: G-CSF water-soluble supernatant with the PotD removed using enterokinase; and

4: G-CSF insoluble inclusion body with the PotD removed using enterokinase.

FIG. 25 shows the results of comparison of the amounts of expression of a recombinant protein fusion-expressed with the RpoA in a water-soluble supernatant (S) and an insoluble inclusion body (IS):

A: RpoA::CUT, RpoA::EGF, RpoA::hFTN-L;

B: RpoA::AID, RpoA::ppGRN; and

C: RpoA;;Nacht, RpoA::G-CSF, RpoA;;GAD₄₄₈₋₅₈₅, RpoA::mp-INS, RpoA::IL-2.

FIG. 26 is a graph comparing the amount of water-soluble expression between a single expression recombinant protein and a recombinant protein fusion-expressed with the RpoA.

FIG. 27 shows the results measuring the resolutions of PNB () and PNP (◯) between a crude water-soluble protein of E. coli, which does not express the cutinase and a crude water-soluble protein of E. coli, which expresses a recombinant protein fusion-expressed with the RpoA in the form of RpoA::CUT.

### BEST MODE

Hereinafter, the present invention will be described in more detail in the following embodiments.

The following embodiments are only intended to illustrate the present invention, but not to limit the spirit and scope of the invention.

### Embodiment 1

### Analysis and Identification of Changes of E. coli Proteins Under Environmental Stresses

### <1-1> Fusion Expression Partner SlyD (FKBP-Type Peptidyl-Prolyl Cis-Trans Isomerase SlyD)

<1-1-1> Collection of Water-Soluble Proteins Expressed in E. coli Cultured Under Thermal Stress

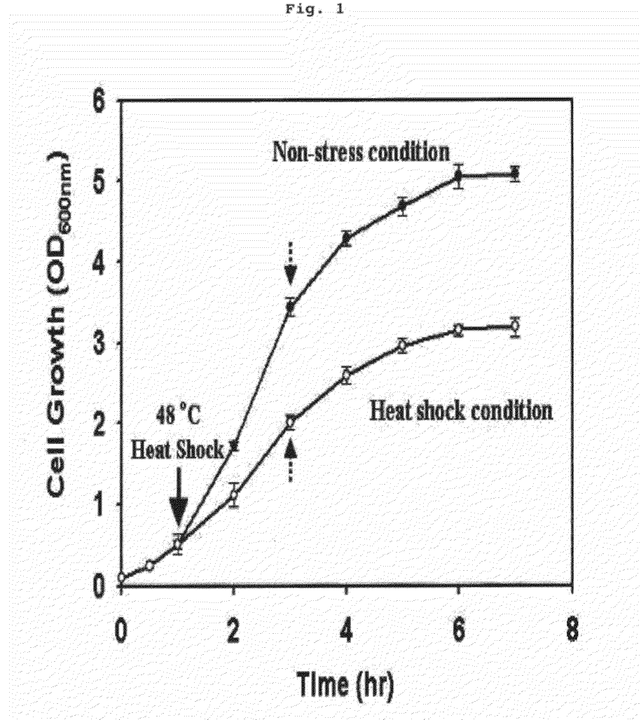

To apply a stress which inhibits the correct folding of proteins in E. coli, a water-soluble protein expressed in E. coli cultured at 48° C. was collected (FIG. 1).

The E. coli BL21 (Escherichia coli K-12) was cultured in the LB medium [10 g Trypton, 10 g Yeast Extract, 5 g NaCl in 1 L water] at 37° C. and 130 rpm. When the OD₆₀₀ is reached at 0.5, the solution was cultured at 37° C. (a control group) and 48° C. (an experimental group) for additional 3 hours and a fungi precipitate was collected by centrifuge of the cell culture media at 4° C., 6000 rpm. The fungi precipitate was washed twice with 40 mM Tris-HCl buffer (pH 8.0), suspended in 500 μl lysis buffer [8 M Urea, 4% (w/v) CHAPS, 40 mM Tris, Protease inhibitor cocktail (Roche Diagnostics GmbH, Germany)], and subsequently lysed using an ultrasonic homogenizer (Branson sonifier, USA). The supernatant and precipitate were separated by centrifuge of the lysed solution at 4° C. and 12,000 rpm for 60 minutes and the removal of the protein inclusion bodies was followed by the separation of the supernatant. The protein concentration of the separated supernatant was measured using a Bio-Rad protein assay kit, and 30 μg water-soluble protein of the supernatant was dissolved in a rehydration solution [2 M thiourea, 8 M urea, 4% (w/v) CHAPS, 1% (w/v) DTT, 1% (w/v) carrier ampholyte, and pH 4.7] and stored at C as a sample for 2-dimensional polyacrylamide gel electrophoresis (2D-PAGE).

### <1-1-2>2-Dimensional Gel Electrophoresis for Analysis of Protein Bodies

The protein bodies of a control group and an experimental group obtained according to the method of the embodiment <1-1-1> were analyzed using a 2-dimensional gel electrophoresis.

The 1-dimensional isoelectric point separation procedure for separation of freeze-stored proteins according to PI was carried out as follows: 45 μg of the freeze-stored proteins were aliquoted into a linear IPG (immobilized pH gradient) gel strip (pH 4-7, 17 cm, ReadyStrip, BIO-RAD, USA) and the separation was carried out on a protein IEF cell electrophoresis apparatus (Bio-Rad Protein IEF cell electrophoresis system, USA) at 500 V for 2 hours; at 1,000 V for 30 minutes; at 2,000 V for 30 minutes, 4,000 V for 30 minutes; at 8,000 V for 70,000 VHr (Volt-hours). The IPG gel strip containing proteins separated according to PI was allowed to react with an equilibration solution [50 mM Tris, pH 8.6, 6 M urea, 30% (v/v) glycerol, 2% SDS] containing 1% DTT and bromophenol blue for 15 minutes and with an equilibration solution containing 2.5% iodoacetamide for additional 15 minutes. To separate proteins according to molecular weights, the 2-dimensional electrophoresis was carried out on the equilibrated gel strips using 12.5% polyacrylamide gel (PROTEAN II Xi cell system (Bio-Rad, USA)). The electrophoresis was carried out at 4° C. until the bromophenol blue reagent is reached at the end of the gel (30 mA/gel, 12 h).

The gel was silver-stained according to the Rabilloud method (Rabilloud T, Methods Mol Biol, 112:297-305, 1999) and scanned using a UMAX PowerLook 1100 Scanner (UMAX, USA). The changes in density per protein spot area on the gel were measured and analyzed using the ImageMaster Software Version 4.01 (Amersham Biosciences, USA).

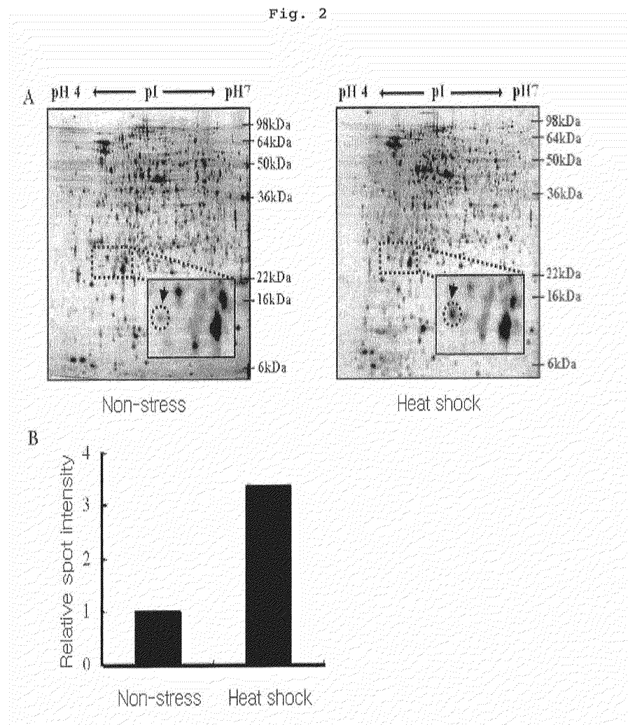

Based on the analysis results, the amount of expression of proteins from the control group was compared with that from the experimental group, and a protein spot with an increase in spot volume by 3.37 times was selected (FIG. 2).

<1-1-3> Identification of the E. coli Proteins with the Amount of Water-Soluble Expression Increased Under Environmental Stresses Using a MALDI-TOF-MS Analysis

A protein spot selected according to the embodiment <1-1-2> was extracted and deposited to the Korea Basic Science Institute, and then identified using a MALDI-TOF (Matrix-Assisted Laser Desorption/Ionization-Time Of Flight)-MS analysis.

Specifically, a protein spot for the MALDI-TOF-MS analysis selected according to the embodiment <1-1-2> was extracted from the silver-stained gel (Gharandaghi F, et al., Electrophoreis, 20:601-605, 1999). The peptide degradation procedure was carried out by incubating the extracted protein spot in 25 nM ammonium bicarbonate solution (pH 8.0) containing 10.15 mg/ml trypsin overnight at 37° C. The degraded peptide was extracted using a 5% (v/v) TFA and a 50% (v/v) ACN solution, and after repeating the extraction procedure three times, a solution containing the peptide extracted using a vacuum centrifuge was dried. The dried peptide was dissolved in a 50% ACN/0.1% TFA solution, deposited to the Korea Basic Science Institute, and its molecular weight was measured using the MALDI-TOF-MS system (Voyager DE-STR instrument; Biosystems, USA). The measured peptide mass fingerprints were carried out using the MS-FIT (http://prospector.ucsf.edu/prospector/4.0.8/html/msfit.htm) of the Prospector website and the Swiss-Prot was used as an MS-FIT database for identification of proteins. Through the protein identification, it was confirmed that the protein with an increase in the amount of expression at 48° C. by 3.37 times was the E. coli SlyD (Table 1).

TABLE 1
Protein Information

| | | | | | Increse | |
|---|---|---|---|---|---|---|
| | | | | | in The | |
| | | | | | relative | |
| | | | | | amount | Se- |
| | Gene | | | | of | quence |
| Gene | accession | Protein | Isoelectric point (pI)/ | | expres- | simi- |
| name | number^{a} | name | molecular weight (kDa) | | sion^{d} | larity |
| SlyD | POA9K9 | FKBP-type | theoretica1 | experimental | 3.37 | 19% |
| | | peptidyl- | value^{b} | value^{c} | | |
| | | prolylcis- | 4.86/20.85 | 4.80/22.23 | | |
| | | trans | | | | |
| | | isomerase | | | | |
| | | SlyD | | | | |

^{a}GenBank Accession No: Identification No. for a gene information search at ExPASy Proteomics Server (http://www.expasy.org/).
^{b}The theoretical values were collected using a Compute pI/Mw tool (http://www.expasy.org/tools/pi_tool.html).
^{c}The experimental values were calculated from 2-D electrophoresis gel images.
^{d}The relative amount of expression of the SlyD in the experimental group was indicated, setting the amount of the SlyD in the control group as 1.

In addition, 98% solubility was shown, in case of the SlyD through a prediction program of the water-soluble proteins (Davis et al., Biotechnol Bioeng 65:382-388, 1999).

### <1-2> Fusion Expression Partner Crr (Glucose-Specific Phosphotransferase Enzyme IIA Component)

<1-2-1> Collection of Water-Soluble Proteins Expressed in E. coli Cultured Under Proteolytic Stress

To apply a stress which inhibits the correct folding of proteins in E. coli, a water-soluble protein expressed in the E. coli in a medium supplemented with GdnHCl, exhibiting the proteolytic effect was collected (FIG. 1).

The E. coliBL21 (Escherichia coli K-12) was cultured in the LB medium [10 g Trypton, 10 g Yeast Extract, 5 g NaCl in 1 L water] at 37° C. and 130 rpm. When the OD₆₀₀ is reached at 0.5, the solution was cultured in a new LB medium (a control group) and a LB medium containing 100 mM GdnHCl (an experimental group) for additional 3 hours and a fungi precipitate was collected by centrifuge of the cell culture media at 4° C., 6000 rpm. The fungi precipitate was washed twice with 40 mM Tris-HCl buffer (pH 8.0), suspended in 500 μl lysis buffer [8 M Urea, 4% (w/v) CHAPS, 40 mM Tris, Protease inhibitor cocktail (Roche Diagnostics GmbH, Germany)], and subsequently lysed using an ultrasonic homogenizer (Branson sonifier, USA). The supernatant and precipitate were separated by centrifuge of the lysed solution at 4° C. and 12,000 rpm for 60 minutes and the removal of the protein inclusion bodies was followed by the separation of the supernatant. The protein concentration of the separated supernatant was measured using a Bio-Rad protein assay kit, and 30 mg water-soluble protein of the supernatant was dissolved in a rehydration solution [2 M thiourea, 8 M urea, 4% (w/v) CHAPS, 1% (w/v) DTT, 1% (w/v) carrier ampholyte, and pH 4.7] and stored at −80° C. as a sample for 2-dimensional polyacrylamide gel electrophoresis (2D-PAGE).

### <1-2-2>2-Dimensional Gel Electrophoresis for Analysis of Protein Bodies

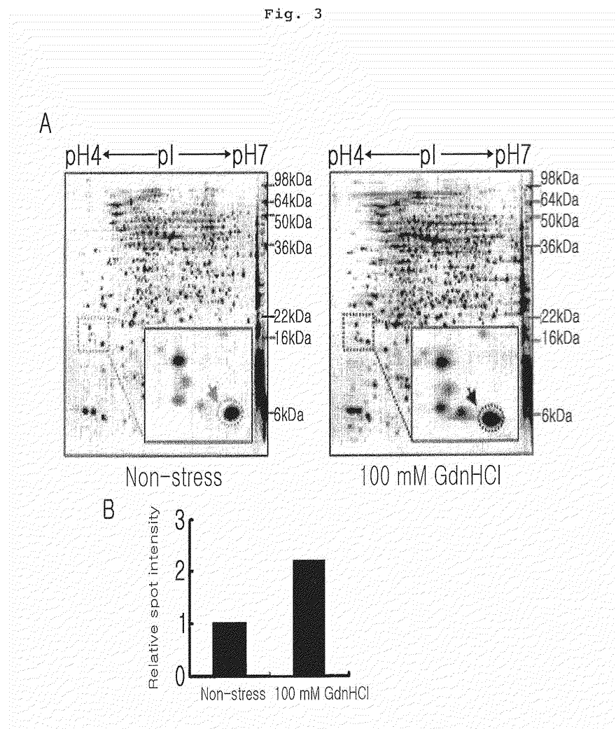

The protein bodies of a control group and an experimental group obtained according to the method of the embodiment <1-2-1> were analyzed using a 2-dimensional gel electrophoresis. The 2-dimensional gel electrophoresis was carried out in the same method as in the embodiment <1-1-2>. Based on the analysis results, the amount of expression of proteins from the control group was compared with that from the experimental group, and a protein spot with an increase in spot volume by 3.37 times was selected (FIG. 3).

<1-2-3> Identification of the E. coli Proteins with the Amount of Water-Soluble Expression Increased Under Environmental Stresses Using a MALDI-TOF-MS Analysis

A protein spot selected according to the embodiment <1-2-2> was extracted and deposited to the Korea Basic Science Institute, and then identified using a MALDI-TOF (Matrix-Assisted Laser Desorption/Ionization-Time Of Flight)-MS analysis.

The identification method was carried out in the same method as in the embodiment <1-1-3>.

Through the protein identification, it was confirmed that the protein with an increase in the amount of expression under the 100 mM GdnHCl by 2.2 times was the E. coli Crr (Table 2).

TABLE 2
Protein Information

| | | | | | Increse | |
|---|---|---|---|---|---|---|
| | | | | | in The | |
| | | | | | relative | |
| | Gene | | | | amount | |
| Gene | accession | Protein | Isoelectric point(pI)/ | | of | Sequence |
| name | number^{a} | name | molecular weight(kDa) | | expression^{d} | similarity |
| Crr | P69783 | Glucose-specific | theoretical | experimental | 2.2 | 26.3% |
| | | phosphotransferase | value^{b} | value^{c} | | |
| | | enzyme IIA | 4.73/18.12 | 4.70/20.08 | | |
| | | component | | | | |

^{a}GenBank Accession No: Identification No. for a gene information search at ExPASy Proteomics Server (http://www.expasy.org/).
^{b}The theoretical values were collected using a Compute pI/Mw tool (http://www.expasy.org/tools/pi_tool.html).
^{c}The experimental values were calculated from 2-D electrophoresis gel images.
^{d}The relative amount of expression of the Crr in the experimental group was indicated, setting the amount of the Crr in the control group as 1.

### <1-3> Fusion Expression Partner RpoS (RNA Polymerase Sigma Factor)

<1-3-1> Collection of Water-Soluble Proteins Expressed in E. coli Cultured Under Proteolytic Stress

To apply a stress which inhibits the correct folding of proteins in E. coli, a water-soluble protein expressed in the E. coli in a medium supplemented with GdnHCl, exhibiting the proteolytic effect was collected.

The protein collection method was carried out in the same method as in the embodiment <1-2-1>.

### <1-3-2>2-Dimensional Gel Electrophoresis for Analysis of Protein Bodies

The protein bodies of a control group and an experimental group obtained according to the method of the embodiment <1-3-1> were analyzed using a 2-dimensional gel electrophoresis.

The 2-dimensional gel electrophoresis was carried out in the same method as in the embodiment <1-1-2>.

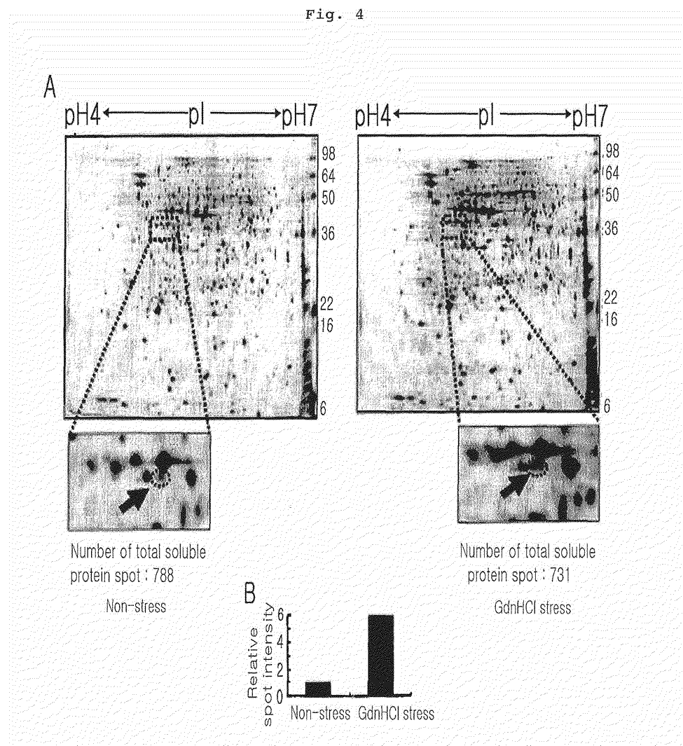

Based on the analysis results, the amount of expression of proteins from the control group was compared with that from the experimental group, and a protein spot with an increase in spot volume by 6 times was selected (FIG. 4).

<1-3-3> Identification of the E. coli Proteins with the Amount of Water-Soluble Expression Increased Under Environmental Stresses Using a MALDI-TOF-MS Analysis

A protein spot selected according to the embodiment <1-3-2> was extracted and deposited to the Korea Basic Science Institute, and then identified using a MALDI-TOF (Matrix-Assisted Laser Desorption/Ionization-Time Of Flight)-MS analysis.

The identification method was carried out in the same method as in the embodiment <1-1-3>.

Through the protein identification, it was confirmed that the protein with an increase in the amount of expression under the 100 mM GdnHCl by 6 times was the RpoS (Table 3).

TABLE 3
Protein Information

| | | | | | Increse | |
|---|---|---|---|---|---|---|
| | | | | | in The | |
| | | | Isoelectric | | relative | Se- |
| | Gene | | point(pI)/ | | amount | quence |
| Gene | accession | Protein | molecular | | of expres- | simi- |
| name | number^{a} | name | weight(kDa) | | sion^{d} | larity |
| RpoS | P13445 | RNA | theo- | experi- | 6 | 18.5% |
| | | polymerase | retical | mental | | |
| | | sigma | value^{b} | value^{c} | | |
| | | factor | 4.9/40.0 | 5.2/39.0 | | |

^{a}GenBank Accession No: Identification No. for a gene information search at ExPASy Proteomics Server (http://www.expasy.org/).
^{b}The theoretical values were collected using a Compute pI/Mw tool (http://www.expasy.org/tools/pi_tool.html).
^{c}The experimental values were calculated from 2-D electrophoresis gel images.
^{d}The relative amount of expression of the RpoS in the experimental group was indicated, setting the amount of the RpoS in the control group as 1.

### <1-4> Fusion Expression Partner PotD (Spermidine/Putrescine-Binding Periplasmic Protein)

<1-4-1> Collection of Water-Soluble Proteins Expressed in E. coli Cultured Under Proteolytic Stress

To apply a stress which inhibits the correct folding of proteins in E. coli, a water-soluble protein expressed in the E. coli in a medium supplemented with GdnHCl, exhibiting the proteolytic effect was collected.

The protein collection method was carried out in the same method as in the embodiment <1-2-1>.

### <1-4-2>2-Dimensional Gel Electrophoresis for Analysis of Protein Bodies

The protein bodies of a control group and an experimental group obtained according to the method of the embodiment <1-4-1> were analyzed using a 2-dimensional gel electrophoresis.

The 2-dimensional gel electrophoresis was carried out in the same method as in the embodiment <1-1-2>.

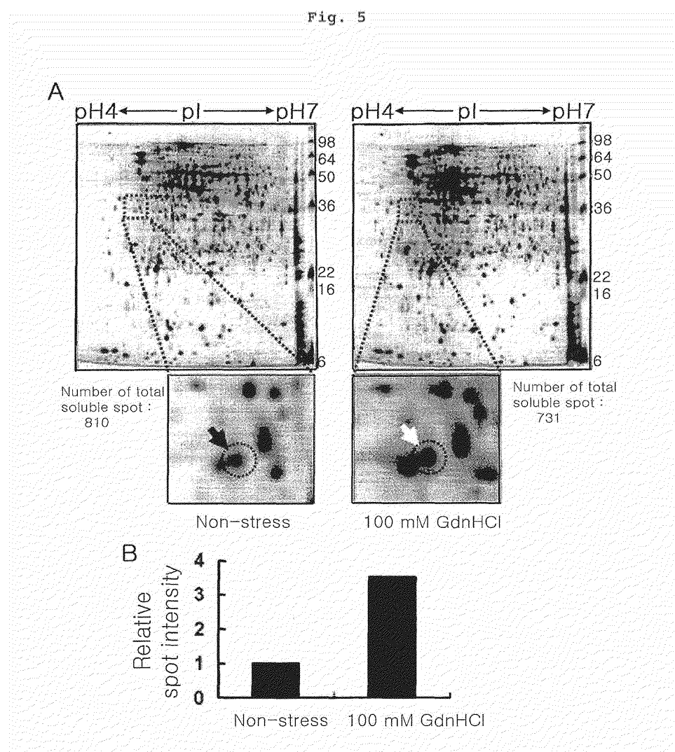

Based on the analysis results, the amount of expression of proteins from the control group was compared with that from the experimental group, and a protein spot with an increase in spot volume by 3.5 times was selected (FIG. 5).

<1-4-3> Identification of the E. coli Proteins with the Amount of Water-Soluble Expression Increased Under Environmental Stresses Using a MALDI-TOF-MS Analysis

A protein spot selected according to the embodiment <1-4-2> was extracted and deposited to the Korea Basic Science Institute, and then identified using a MALDI-TOF (Matrix-Assisted Laser Desorption/Ionization-Time Of Flight)-MS analysis.

The identification method was carried out in the same method as in the embodiment <1-1-3>.

Through the protein identification, it was confirmed that the protein with an increase in the amount of expression under the 100 mM GdnHCl by 3.5 times was the PotD (Table 4).

TABLE 4
Protein Information

| | | | | | Increse | |
|---|---|---|---|---|---|---|
| | | | | | in The | |
| | | | Isoelectric | | relative | Se- |
| | Gene | | point(pI)/ | | amount | quence |
| Gene | accession | Protein | molecular | | of expres- | simi- |
| name | number^{a} | name | weight(kDa) | | sion^{d} | larity |
| PotD | POAFK9 | Spermidine/ | theo- | experi- | 3.5 | 18% |
| | | putrescine- | retical | mental | | |
| | | binding | value^{b} | value^{c} | | |
| | | periplasmic | 4.86/ | 4.85/ | | |
| | | protein | 36.49 | 39.79 | | |

^{a}GenBank Accession No: Identification No. for a gene information search at ExPASy Proteomics Server ( http://www.expasy.org/).
^{b}The theoretical values were collected using a Compute pI/Mw tool (http://www.expasy.org/tools/pi_tool.html).
^{c}The experimental values were calculated from 2-D electrophoresis gel images.
^{d}The relative amount of expression of the RpoS in the experimental group was indicated, setting the amount of the PotD in the control group as 1.

### <1-5> Fusion Expression Partner RNA Polymerase Alpha Subunit (RpoA)

<1-5-1> Collection of Water-Soluble Proteins Expressed in E. coli Cultured Under 2-HEDS Stress

To apply a stress which inhibits the correct folding of proteins in E. coli, a water-soluble protein expressed in E. coli cultured in a medium supplemented with 2-hydroxyethyldisulfide (2-HEDS) was collected.

The E. coliBL21 (Escherichia coli K-12) was cultured in the LB medium [10 g Trypton, 10 g Yeast Extract, 5 g NaCl in 1 L water] at 37° C. and 130 rpm. When the OD₆₀₀ is reached at 0.5, the solution was cultured in a new LB medium (a control group) and a LB medium containing 10 mM 2-HEDS (an experimental group) for additional 3 hours and a fungi precipitate was collected by centrifuge of the cell culture media at 4° C., 6000 rpm. The fungi precipitate was washed twice with 40 mM Tris-HCl buffer (pH 8.0), suspended in 500 μl lysis buffer [8 M Urea, 4% (w/v) CHAPS, 40 mM Tris, Protease inhibitor cocktail (Roche Diagnostics GmbH, Germany)], and subsequently lysed using an ultrasonic homogenizer. The supernatant and precipitate were separated by centrifuge of the lysed solution at 4° C. and 12,000 rpm for 60 minutes and the removal of the protein inclusion bodies was followed by the separation of the supernatant. The protein concentration of the separated supernatant was measured using a Bio-Rad protein assay kit, and 30 mg water-soluble protein of the supernatant was dissolved in a rehydration solution [2 M thiourea, 8 M urea, 4% (w/v) CHAPS, 1% (w/v) DTT, 1% (w/v) carrier ampholyte, and pH 4.7] and stored at −80° C. as a sample for 2-dimensional polyacrylamide gel electrophoresis (2D-PAGE).

### <1-5-2>2-Dimensional Gel Electrophoresis for Analysis of Protein Bodies

The protein bodies of a control group and an experimental group obtained according to the method of the embodiment <1-5-1> were analyzed using a 2-dimensional gel electrophoresis.

The 2-dimensional gel electrophoresis was carried out in the same method as in the embodiment <1-1-2>.

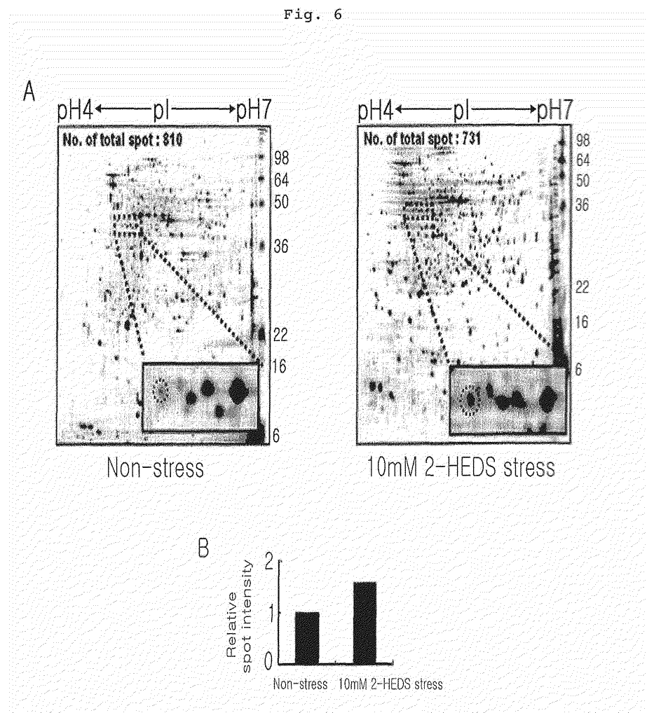

Based on the analysis results, the amount of expression of proteins from the control group was compared with that from the experimental group, and a protein spot with an increase in spot volume by 1.5 times was selected (FIG. 6).

<1-5-3> Identification of the E. coli Proteins with the Amount of Water-Soluble Expression Increased Under Environmental Stresses Using a MALDI-TOF-MS Analysis

A protein spot selected according to the embodiment <1-5-2> was extracted and deposited to the Korea Basic Science Institute, and then identified using a MALDI-TOF (Matrix-Assisted Laser Desorption/Ionization-Time Of Flight)-MS analysis.

The identification method was carried out in the same method as in the embodiment <1-1-3>.

Through the protein identification, it was confirmed that the protein with an increase in the amount of expression under 10 mM 2-HEDS by 1.5 times was the RpoA (Table 5).

TABLE 5
Protein Information

| | | | | | Increse | |
|---|---|---|---|---|---|---|
| | | | | | in The | |
| | | | Isoelectric | | relative | Se- |
| | Gene | | point(pI)/ | | amount | quence |
| Gene | accession | Protein | molecular | | of expres- | simi- |
| name | number^{a} | name | weight(kDa) | | sion^{d} | larity |
| RpoA | POA7Z4 | DNA- | theo- | experi- | 1.5 | 13.4% |
| | | Directed | retical | mental | | |
| | | RNA | value^{b} | value^{c} | | |
| | | polymerase | 4.98/ | 5.06/ | | |
| | | Alpha chain | 36.51 | 39.5 | | |

^{a}GenBank Accession No: Identification No. for a gene information search at ExPASy Proteomics Server (http://www.expasy.org/).
^{b}The theoretical values were collected using a Compute pI/Mw tool (http://www.expasy.org/tools/pi_tool.html).
^{c}The experimental values were calculated from 2-D electrophoresis gel images.
^{d}The relative amount of expression of the RpoS in the experimental group was indicated, setting the amount of the PotD in the control group as 1.

### Embodiment 2

### Preparation Of an Expression Vector Containing a Fusion Expression Partner at the Amino Terminal

### <2-1> Preparation of an Expression Vector Containing the SlyD as a Fusion Expression Partner

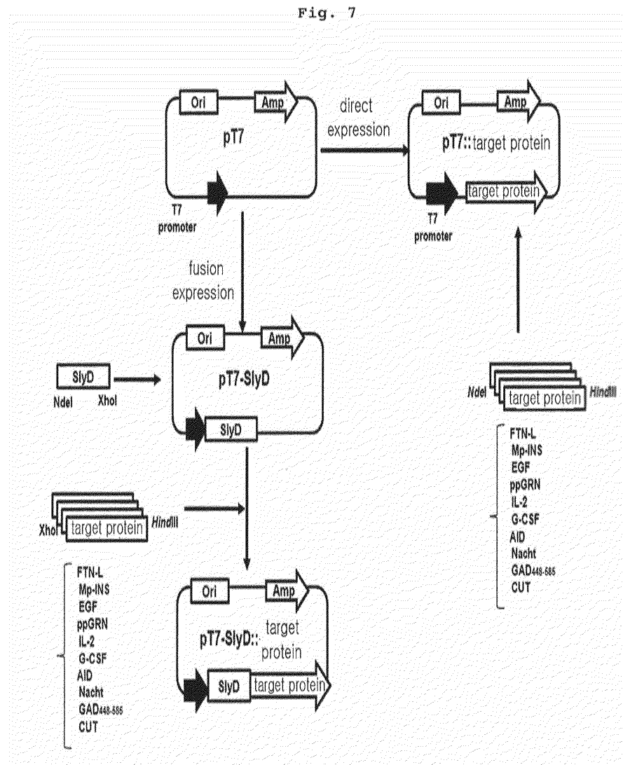

An expression vector containing an E. coli protein SlyD with the amount of water-soluble expression increased under an environmental stress selected according to the method in the embodiment <1-1> as a fusion expression partner was prepared. To obtain a nucleotide encoding the SlyD gene, a pair of primers for PCR amplification of the SlyD gene except for stop codons were prepared using the sequence information (SEQ ID NO. 1) of 3475929 bp to 3476519 bp in gi:49175990 from the Entrez Nucleotide database. In the preparation of the pair of primers, a recognition sequence for the NdeI restriction enzyme and a recognition sequence for the XhoI restriction enzyme were also included in the sense primer (SEQ ID NO. 2: cat atg aaa gta gca aaa gac ctg) and the antisense primer (SEQ ID NO. 3: ctc gag gtg gca acc gca acc gcc gtt), respectively. 100 ng of chromosome separated from the E. coli as a template DNA and 50 pmole of each pair of primers represented by SEQ ID No. 2 and 3 were added to a buffer for DNA polymerase enzyme reaction (0.25 mM dNTPs; 50 mM KCl; 10 mM (NH₄)₂SO₄; 20 mM Tris-HCl (pH8.8); 2 mM MgSO₄; 0.1% Triton X-100), the PCR was then carried out using a Taq DNA polymerase enzyme. The reactions were carried out 30 times in total at the reaction conditions of 95° C./30 sec (degradation), 52° C./30 sec (annealing), and 72° C./60 sec (elongation). As a result, PCR products were obtained, containing the NdeI restriction enzyme site at the 5′ terminal of the amplified DNA fragment and the XhoI restriction enzyme site at the 3′ terminal of the fragment. The amplified PCR products was treated with the restriction enzymes NdeI and XhoI and inserted into the restriction enzyme NdeI and XhoI sites of pT7-7 (Novagen, USA). The insertion is referred to as pT7-SlyD (FIG. 7).

### <2-2> Preparation of an Expression Vector Containing the Crr as a Fusion Expression Partner

An expression vector containing an E. coli protein Crr with the amount of water-soluble expression increased under an environmental stress selected according to the method in the embodiment <1-2> as a fusion expression partner was prepared.

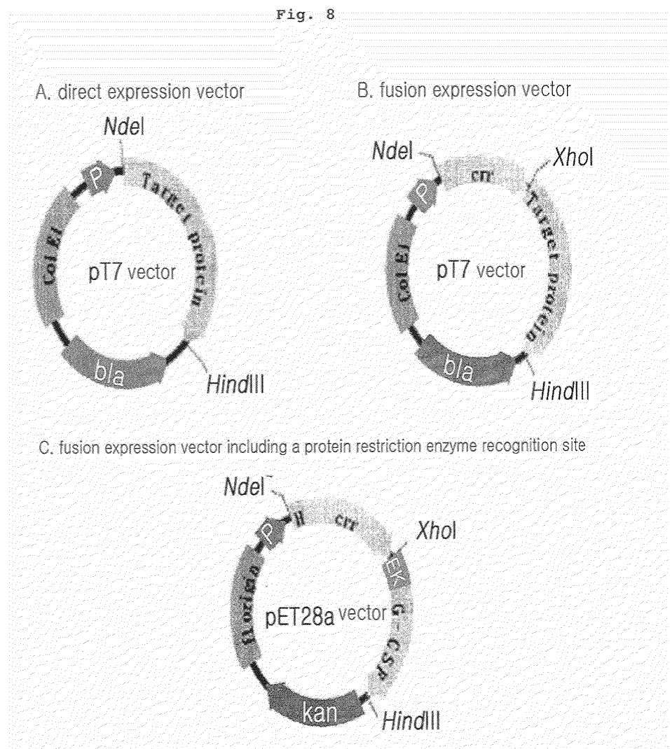

To obtain a nucleotide encoding the Crr gene, a pair of primers for PCR amplification of the Crr gene except for stop codons were prepared using the sequence information (SEQ ID NO. 4) of 2533856 bp to 2534365 bp in gi:49175990 from the Entrez Nucleotide database. In the preparation of the pair of primers, a recognition sequence for the NdeI restriction enzyme and a recognition sequence for the XhoI restriction enzyme were also included in the sense primer (SEQ ID NO. 5: cat atg ggt ttg ttc gat aaa ctg) and the antisense primer (SEQ ID NO. 6: ctc gag ctt ctt gat gcg gat aac), respectively. 100 ng of chromosome separated from the E. coli as a template DNA and 50 pmole of each pair of primers represented by SEQ ID NOs. 5 and 6 were added to a buffer for DNA polymerase enzyme reaction (0.25 mM dNTPs; 50 mM KCl; 10 mM (NH₄)₂SO₄; 20 mM Tris-HCl (pH8.8); 2 mM MgSO₄; 0.1% Triton X-100), the PCR was then carried out using a Taq DNA polymerase enzyme. The reactions were carried out 30 times in total at the reaction conditions of 95° C./30 sec (degradation), 52° C./30 sec (annealing), and 72° C./60 sec (elongation). As a result, PCR products were obtained, containing the NdeI restriction enzyme site at the 5′ terminal of the amplified DNA fragment and the XhoI restriction enzyme site at the 3′ terminal of the fragment. The amplified PCR products was treated with the restriction enzymes NdeI and XhoI and inserted into the restriction enzyme NdeI and XhoI sites of pT7-7 (Novagen, USA). The insertion is referred to as pT7-Crr (FIG. 8).

### <2-3> Preparation of an Expression Vector Containing the RpoS as a Fusion Expression Partner

An expression vector containing an E. coli protein RpoS with the amount of water-soluble expression increased under an environmental stress selected according to the method in the embodiment <1-3> as a fusion expression partner was prepared.

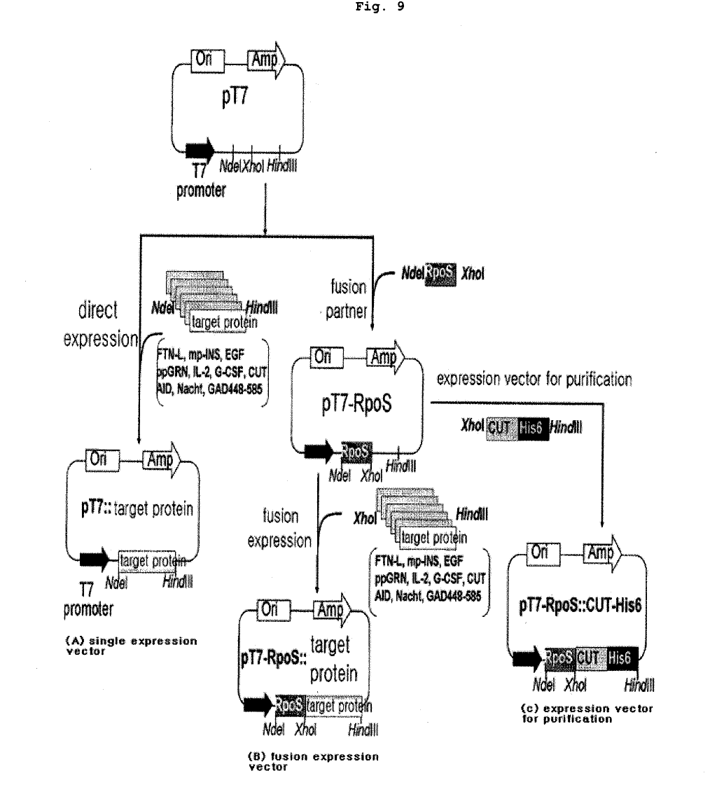

To obtain a nucleotide encoding the RpoS gene, a pair of primers for PCR amplification of the RpoS gene except for stop codons were prepared using the sequence information (SEQ ID NO. 7) of 2864581 bp to 2865573 bp in gi:49175990 from the Entrez Nucleotide database. In the preparation of the pair of primers, a recognition sequence for the NdeI restriction enzyme and a recognition sequence for the XhoI restriction enzyme were also included in the sense primer (SEQ ID NO. 8: cat atg agt cag aat acg ctg aaa) and the antisense primer (SEQ ID NO. 9: ctc gag ctc gcg gaa cag cgc ttc), respectively. 100 ng of chromosome separated from the E. coli as a template DNA and 50 pmole of each pair of primers represented by SEQ ID NOs. 8 and 9 were added to a buffer for DNA polymerase enzyme reaction (0.25 mM dNTPs; 50 mM KCl; 10 mM (NH₄)₂SO₄; 20 mM Tris-HCl (pH8.8); 2 mM MgSO₄; 0.1% Triton X-100), the PCR was then carried out using a Taq DNA polymerase enzyme. The reactions were carried out 30 times in total at the reaction conditions of 95° C./30 sec (degradation), 52° C./30 sec (annealing), and 72° C./60 sec (elongation). As a result, PCR products were obtained, containing the NdeI restriction enzyme site at the 5′ terminal of the amplified DNA fragment and the XhoI restriction enzyme site at the 3′ terminal of the fragment. The amplified PCR products was treated with the restriction enzymes NdeI and XhoI and inserted into the restriction enzyme NdeI and XhoI sites of pT7-7 (Novagen, USA). The insertion is referred to as pT7-RpoS (FIG. 9).

### <2-4> Preparation of an Expression Vector Containing the PotD as a Fusion Expression Partner

An expression vector containing an E. coli protein PotD with the amount of water-soluble expression increased under an environmental stress selected according to the method in the embodiment <1-4> as a fusion expression partner was prepared.

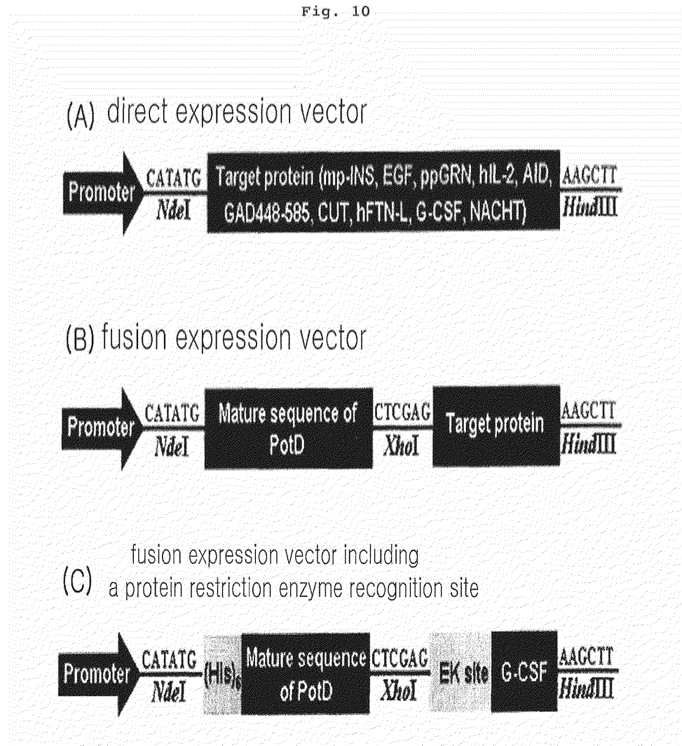

To obtain a nucleotide encoding the PotD gene, a pair of primers for PCR amplification of the PotD gene except for stop codons were prepared using the sequence information (SEQ ID NO. 10) of 1181006 bp to 1182052 bp in gi:49175990 from the Entrez Nucleotide database. In the preparation of the pair of primers, a recognition sequence for the NdeI restriction enzyme and a recognition sequence for the XhoI restriction enzyme were also included in the sense primer (SEQ ID NO. 11: cat atg aaa gtc gca gtc ctc ggc) and the antisense primer (SEQ ID NO. 12: ctc gag acg tcc tgc ttt cag ctt), respectively. 100 ng of chromosome separated from the E. coli as a template DNA and 50 pmole of each pair of primers represented by SEQ ID NOs. 11 and 12 were added to a buffer for DNA polymerase enzyme reaction (0.25 mM dNTPs; 50 mM KCl; 10 mM (NH₄)₂SO₄; 20 mM Tris-HCl (pH8.8); 2 mM MgSO₄; 0.1% Triton X-100), the PCR was then carried out using a Taq DNA polymerase enzyme. The reactions were carried out 30 times in total at the reaction conditions of 95° C./30 sec (degradation), 52° C./30 sec (annealing), and 72° C./60 sec (elongation). As a result, PCR products were obtained, containing the NdeI restriction enzyme site at the 5′ terminal of the amplified DNA fragment and the XhoI restriction enzyme site at the 3′ terminal of the fragment. The amplified PCR products was treated with the restriction enzymes NdeI and XhoI and inserted into the restriction enzyme NdeI and XhoI sites of pT7-7 (Novagen, USA). The insertion is referred to as pT7-PotD (FIG. 10).

### <2-5> Preparation of an Expression Vector Containing the RpoA as a Fusion Expression Partner

An expression vector containing an E. coli protein RNA polymerase α subunit (RpoA) with the amount of water-soluble expression increased under an environmental stress selected according to the method in the embodiment <1-5> as a fusion expression partner was prepared.

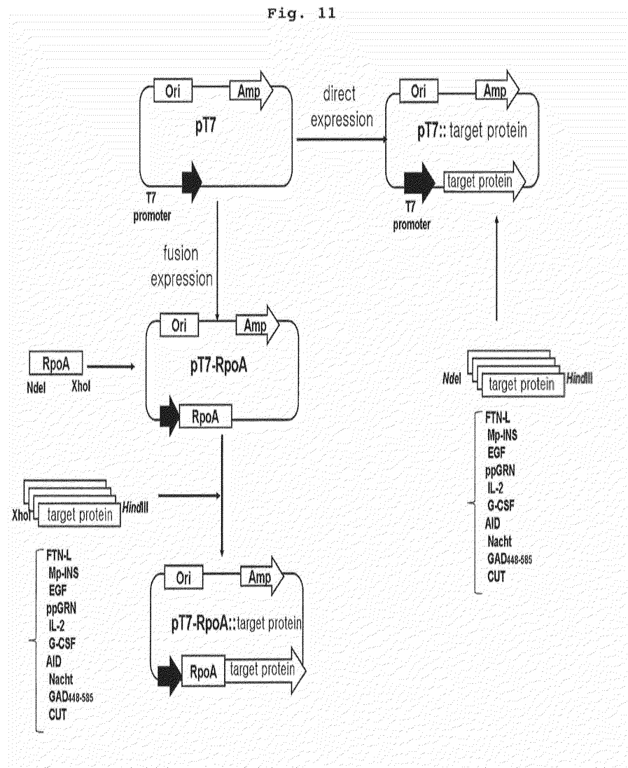

To obtain a nucleotide encoding the RpoA gene, a pair of primers for PCR amplification of the RpoA gene except for stop codons were prepared using the sequence information (SEQ ID NO. 13) of 3438062 bp to 3439051 bp in gi:49175990 from the Entrez Nucleotide database. In the preparation of the pair of primers, a recognition sequence for the NdeI restriction enzyme and a recognition sequence for the XhoI restriction enzyme were also included in the sense primer (SEQ ID NO. 14: cat atg cag ggt tct gtg aca gag) and the antisense primer (SEQ ID NO. 15: ctc gag tta ctc gtc agc gat get tgc), respectively. 100 ng of chromosome separated from the E. coli as a template DNA and 50 pmole of each pair of primers represented by SEQ ID NOs. 14 and 15 were added to a buffer for DNA polymerase enzyme reaction (0.25 mM dNTPs; 50 mM KCl; 10 mm (NH₄)₂SO₄; 20 mM Tris-HCl (ph8.8); 2 mM MgSO₄; 0.1% Triton x-100), the PCR was then carried out using a Taq DNA polymerase enzyme. The reactions were carried out 30 times in total at the reaction conditions of 95° C./30 sec (degradation), 52° C./30 sec (annealing), and 72° C./60 sec (elongation). As a result; PCR products were obtained, containing the NdeI restriction enzyme site at the 5′ terminal of the amplified DNA fragment and the XhoI restriction enzyme site at the 3′ terminal of the fragment. The amplified PCR products was treated with the restriction enzymes NdeI and XhoI and inserted into the restriction enzyme NdeI and XhoI sites of pT7-7 (Novagen, USA). The insertion is referred to as pT7-RpoA (FIG. 11).

### Embodiment 3

### Preparation of an Expression Vector for a Heterologous in the Form of a Fusion Protein

### <3-1> Preparation of a Single Expression Vector for a Heterologous Protein

A heterologous protein is one selected from the group consisting of human minipro-insulin (hereinafter, referred to as “mp-INS”; EF518215:1-180 bp; SEQ ID NO. 16), human epidermal growth factor (hereinafter, referred to as “EGF”; NCBI Nucleotide accession number M15672: 1-165 bp), human prepro-ghrelin, hereinafter, referred to as “ppGRN”; NCBI Nucleotide accession number NM;016362: 109-393), human interleukin-2 (hereinafter, referred to as “hIL-2”; NCBI Nucleotide accession number NM;000586: 116-517), human activation induced cytidine deaminase (hereinafter, referred to as “AID”; NCBI Nucleotide accession number NM;020661: 77-673 bp), human glutamate decarboxylase (hereinafter, referred to as “GAD₄₄₈₋₅₈₅”; SEQ ID NO. 17), cutinase (hereinafter, referred to as “CUT”; SEQ ID NO. 18) derived from Pseudomonas putida, human ferritin light chain (hereinafter, referred to as “hFTN-L”; NM;00146:200-727 bp; SEQ ID NO. 19), human granulocyte colony-stimulating factor (hereinafter, referred to as “G-CSF”; NCBI Nucleotide accession number NM;172219: 131-655 bp), and cold autoinflammatoryo syndromel (NALP3) Nacht domain (hereinafter, referred to as “Nacht”; SEQ ID NO. 20). A pair of primers in Table 6 prepared to include a recognition sequence for the NdeI restriction enzyme at the 5′-terminal of the heterologous protein and a recognition sequence for the HindIII at the 3′-terminal of the protein were added to a buffer for DNA polymerase reaction (0.25 mM dNTPs; 50 mM KCl; 10 mM (NH₄)₂SO₄; 20 mM Tris-HCl (pH8.8); 2 mM MgSO₄; 0.1% Triton X-100) and 100 ng of DNA as a template. The PCRs were then carried out 30 times in total at the reaction conditions of 95° C./30 sec (degradation), 52° C./30 sec (annealing), and 72° C./60 sec (elongation) using a Taq DNA polymerase enzyme.

Specifically, the amino acid sequences containing the stop codons except for the start codons were amplified in mp-INS, EGF, ppGRN, hIL-2, AID, CUT, G-CSF, and hFTN-L. 138 amino acid sequences (SEQ ID NO. 17) corresponding to those from 448th to 585th of the GAD₄₄₈₋₅₈₅ and 316 amino acid sequences (SEQ ID NO. 20) corresponding to the nacht domain of the cryopyrin of Nacht. Then, a primer was prepared to contain a recognition sequence for the ClaI restriction enzyme at the 3′-terminal of GAD₄₄₈₋₅₈₅. In addition, a template DNA was one selected from mp-INS, EGF, ppGRN, hIL-2, AID, GAD₄₄₈₋₅₈₅, hFTN-L, G-CSF, and Nacht. The whole RNA was extracted from a human tissue, in which the proteins were quite often expressed, using a RNeasy mini kit (QIAGEN, USA). 1 μg of the whole RNA and 1 μl oligo-d (T) was filled with 50 μl of DI water and was allowed to carry out a RT-PCR (reverse transcription polymerase chain reaction). The cDNAs were obtained from the reactions at 70° C. for 5 minutes, at 4° C. for 5 minutes, at 42° C. for 60 minutes, at 94° C. for 5 minutes, and at 4° C. for 5 minutes. The mp-INS was cloned in a human pancreatic tissue, the EGF in an epithelial cell, the ppGRN in a human placenta, the hIL-2, hFTN-L, G-CSF, and Nacht in a human leukocyte, the AID in a human temporal lobe, and the GAD₄₄₈₋₅₈₅ in a human hippocampus tissue, respectively. In case of CUT, a DNA extracted using a Genomic DNA Purification kit (promega, USA) from Pseudomonas putida was used as a template DNA.

TABLE 6
Primer Sequences

| Gene Name | Primer | SEQ ID NO | Base sequence |
|---|---|---|---|
| mp-INS | Sense | SEQ ID NO. 21 | cat atg ttt gtc aac caa cat |
| | antisense | SEQ ID NO. 22 | aat ctt tta gtt aca gta gtt c |
| EGF | Sense | SEQ ID NO. 23 | cat atg aac tct gac tcc gaa tgc |
| | antisense | SEQ ID NO. 24 | aag ctt tta acg cag ttc cca cca |
| ppGRN | Sense | SEQ ID NO. 25 | cat atg ggc tcc agc ttc ctg |
| | antisense | SEQ ID NO. 26 | aag ctt tca ctt gtc ggc t |
| hIL-2 | Sense | SEQ ID NO. 27 | cat atg gca cct act tca agt |
| | antisense | SEQ ID NO. 28 | aag ctt tta tca agt cag tgt |
| AID | Sense | SEQ ID NO. 29 | cat atg gac agc ctc ttg atg aac |
| | antisense | SEQ ID NO. 30 | aag ctt tca taa caa aag tcc ca |
| GAD₄₄₈₋₅₈₅ | Sense | SEQ ID NO. 31 | cat atg cgc cac gtt gat gt |
| | antisense | SEQ ID NO. 32 | atc gat tta taa atc ttg tcc |
| CUT | Sense | SEQ ID NO. 33 | cat atg gct ccc ctg ccg gat ac |
| | antisense | SEQ ID NO. 34 | aag ctt tta aag ccc gcg gcg ct |
| hFTN-L | Sense | SEQ ID NO. 35 | cat atg agc tcc cag att cgt |
| | antisense | SEQ ID NO. 36 | aag ctt tta gtc gtg ctt gag agt |
| G-CSF | Sense | SEQ ID NO. 37 | cat atg act cca ctc gga cct g |
| | antisense | SEQ ID NO. 38 | aag ctt tca tgg ctg tgc aag |
| Nacht | Sense | SEQ ID NO. 39 | cat atg act gtg gtg ttc cag |
| | antisense | SEQ ID NO. 40 | aag ctt tca cag cag gta gta c |

The PCR amplification products were treated with the NdeI/HindIII (NdeI/ClaI in case of GAD₄₄₈₋₅₈₅) restriction enzyme and then inserted into the NdeI/HindIII (NdeI/ClaI in case of GAD₄₄₈₋₅₈₅) to obtain a single expression vector of the heterologous protein.

<3-2> Preparation of a Fusion Expression Vector with a Fusion Expression Partner of the Heterologous Protein
<3-2-1> Preparation of a Fusion Expression Vector of the Heterologous Protein with its Fusion Expression Partner SlyD

100 ng of template DNA was added to each of 50 pmole of buffers for DNA polymerase reaction (0.25 mM dNTPs; 50 mM KCl; 10 mM (NH₄)₂SO₄; 20 mM Tris-HCl (pH8.8); 2 mM MgSO₄; 0.1% Triton X-100) including a pair of primers in Table 7 prepared to contain a recognition sequence for the XhoI restriction enzyme at the 5′-terminal of the heterologous, such as mp-INS, EGF, ppGRN, hIL-2, AID, GAD₄₄₈₋₅₈₅, CUT, hFTN-L, G-CSF, and Nacht, and a recognition sequence for the HindIII at the 3′-terminal of the protein. The PCRs were then carried out 30 times in total at the reaction conditions of 95° C./30 sec (degradation), 52° C./30 sec (annealing), and 72° C./60 sec (elongation) using a Taq DNA polymerase enzyme.

Specifically, the amino acid sequences containing the stop codons except for the start codons were amplified in hFTN-L, mp-INS, EGF, ppGRN, hIL-2, AID, and CUT. 138 amino acid sequences (SEQ ID NO. 17) corresponding to those from 448th to 585th of the GAD₄₄₈₋₅₈₅ and 316 amino acid sequences (SEQ ID NO. 20) corresponding to the nahct domain of the cryopyrin of Nacht. Then, a primer was prepared to contain a recognition sequence for the ClaI restriction enzyme at the 3′-terminal of GAD₄₄₈₋₅₈₅.

TABLE 7
Primer Sequence

| Gene Name | Primer | SEQ ID NO. | Base Sequence |
|---|---|---|---|
| mp-INS | Sense | SEQ ID NO. 41 | ctc gag ttt gtc aac caa cat |
| | Antisense | SEQ ID NO. 42 | aag ctt tta gtt aca gta gtt c |
| EGF | Sense | SEQ ID NO. 43 | ctc gag aac tct gac tcc gaa tgc |
| | Antisense | SEQ ID NO. 44 | aag ctt tta acg cag ttc cca cca |
| ppGRN | Sense | SEQ ID NO. 45 | ctc gag ggc tcc agc ttc ctg |
| | Antisense | SEQ ID NO. 46 | aag ctt tca ctt gtc ggc t |
| hIL-2 | Sense | SEQ ID NO. 47 | ctc gag gca cct act tca agt |
| | Antisense | SEQ ID NO. 48 | aag ctt tta tca agt cag tgt |
| AID | Sense | SEQ ID NO. 49 | ctc gag gac agc ctc ttg atg aac |
| | Antisense | SEQ ID NO. 50 | aag ctt tca taa caa aag tcc ca |
| GAD₄₄₈₋₅₈₅ | Sense | SEQ ID NO. 51 | ctc gag cgc cac gtt gat gt |
| | Antisense | SEQ ID NO. 52 | atc gat tta taa atc ttg tcc |
| CUT | Sense | SEQ ID NO. 53 | ctc gag gct ccc ctg ccg gat ac |
| | Antisense | SEQ ID NO. 54 | aag ctt tta aag ccc gcg gcg ct |
| hFTN-L | Sense | SEQ ID NO. 55 | ctc gag agc tcc cag att cgt |
| | Antisense | SEQ ID NO. 56 | aag cct tta gtc gtg ctt gag agt |
| G-CSF | Sense | SEQ ID NO. 57 | ctc gag act cca ctc gga cct g |
| | Antisense | SEQ ID NO. 58 | aag ctt tca tgg ctg tgc aag |
| Nacht | Sense | SEQ ID NO. 59 | ctc gag act gtg gtg ttc cag |
| | Antisense | SEQ ID NO. 60 | aag ctt tca cag cag gta gta c |

The PCR amplification products were treated with the XhoI/HindIII (NdeI/ClaI in case of GAD₄₄₈₋₅₈₅) restriction enzyme and then inserted into the site of the XhoI/HindIII prepared by the method in the embodiment <2-1> (NdeI/ClaI in case of GAD₄₄₈₋₅₈₅) to obtain a single expression vector of the heterologous protein. The plasmids prepared by the method were referred to as pT7-SlyD::FTN-L, pT7-SlyD::IL-2, pT7-SlyD::EGF, pT7-SlyD::G-CSF, pT7-SlyD::AID, pT7-SlyD::CUT, pT7-SlyD::ppGRN, pT7-SlyD::GAD₄₄₈₋₅₈₅, and pT7-SlyD::Nacht, respectively (FIG. 7).

<3-2-2> Preparation of a Fusion Expression Vector of the Heterologous Protein with its Fusion Expression Partner Crr

The PCR was carried out in the same method as in the embodiment <3-2-2>.

The PCR amplification products were treated with the XhoI/HindIII (NdeI/ClaI in case of GAD₄₄₈₋₅₈₅) restriction enzyme and then inserted into the site of the XhoI/HindIII prepared by the method in the embodiment <2-2> (NdeI/ClaI in case of GAD₄₄₈₋₅₈₅) to obtain a single expression vector of the heterologous protein. The plasmids prepared by the method were referred to aspT7-Crr::FTN-L, pT7-Crr::IL-2, pT7-Crr::EGF,pT7-Crr::G-CSF, pT7-Crr::AID, pT7-Crr::CUT, pT7-Crr::ppGRN, pT7-Crr::GAD₄₄₈₋₅₈₅, and pT7-Crr::Nacht, respectively.

<3-2-3> Preparation of a Fusion Expression Vector of the Heterologous Protein with its Fusion Expression Partner RpoS

The PCR was carried out in the same method as in the embodiment <3-2-2>.

The PCR amplification products were treated with the XhoI/HindIII (XhoI/ClaI in case of GAD₄₄₈₋₅₈₅) restriction enzyme and then inserted into the site of the XhoI/HindIII of the pT7-RpoS prepared by the method in the embodiment <2-3> (NdeI/ClaI in case of GAD₄₄₈₋₅₈₅) to obtain a single expression vector of the heterologous protein. The plasmids prepared by the method were referred to as pT7-RpoS::FTN-L, pT7-RpoS::IL-2, pT7-RpoS::EGF, pT7-Rpos::G-CSF, pT7-RpoS::AID, pT7-RpoS::CUT, pT7-RpoS::ppGRN, pT7-RpoS::GAD₄₄₈₋₅₈₅, and pT7-RpoS::Nacht, respectively (FIG. 9).

<3-2-4> Preparation of a Fusion Expression Vector of the Heterologous Protein with its Fusion Expression Partner PotD

The PCR was carried out in the same method as in the embodiment <3-2-2>.

The PCR amplification products were treated with the XhoI/HindIII (XhoI/ClaI in case of GAD₄₄₈₋₅₈₅) restriction enzyme and then inserted into the site of the XhoI/HindIII of the pT7-PotD prepared by the method in the embodiment <2-4> (NdeI/ClaI in case of GAD₄₄₈₋₅₈₅) to obtain a single expression vector of the heterologous protein. The plasmids prepared by the method were referred to as pT7-PotD::FTN-L, pT7-PotD::IL-2, pT7-PotD::EGF, pT7-PotD::G-CSF, pT7-PotD::AID, pT7-PotD::CUT, pT7-PotD::ppGRN, pT7-PotD::GAD₄₄₈₋₅₈₅, and pT7-PotD::Nacht, respectively.

<3-2-5> Preparation of a Fusion Expression Vector of the Heterologous Protein with its Fusion Expression Partner RpoA

The PCR was carried out in the same method as in the embodiment <3-2-2>.

The PCR amplification products were treated with the XhoI/HindIII (XhoI/ClaI in case of GAD₄₄₈₋₅₈₅) restriction enzyme and then inserted into the site of the XhoI/HindIII of the pT7-RpoA prepared by the method in the embodiment <2-5> (NdeI/ClaI in case of GAD₄₄₈₋₅₈₅) to obtain a single expression vector of the heterologous protein. The plasmids prepared by the method were referred to as pT7-RpoA::FTN-L, pT7-RpoA::IL-2, pT7-RpoA::EGF, pT7-RpoA::G-CSF, pT7-RpoA::AID, pT7-RpoA::CUT, pT7-RpoA::ppGRN, pT7-RpoA::GAD₄₄₈₋₅₈₅, and pT7-RpoA::Nacht, respectively (FIG. 11).

### Embodiment 4

### Water-Soluble Expression of a Recombinant Protein

A single expression vector of the heterologous protein prepared by the method of the embodiment <3> and a fusion expression vector with the fusion expression partner were transformed in E. coli and cultured, and the effects of the water-soluble expression by a fusion expression partner of the present invention was confirmed by inducing the expression of a recombinant protein with the IPTG.

<4-1> Confirmation of the Water-Soluble Expression of the Heterologous Protein Fused with the Fusion Expression Partner SlyD
<4-1-1> Transformation in E. coli and Expression of a Recombinant Protein

The vectors prepared in the embodiments <3-1> and <3-2-2> were transformed in E. coli, using the method described by Hanahan (Hanahan D, DNA Cloning vol. 1 109-135, IRS press 1985).

Specifically, the vectors in the embodiments <3-1> and <3-2-1> were transformed in the E. coli BL21 (DE3) treated with CaCl₂ using the thermal shock method, and a colony of the expression vectors, which were transformed due to the growth in a culture medium containing ampicillin and exhibited the ampicillin resistance, were screened. A E. coli transformed with a fusion expression vector pT7-SlyD (pT7-SlyD::mp-INS, pT7-RpoA::FTN-L, pT7-RpoA::IL-2, pT7-RpoA::EGF, pT7-RpoA::G-CSF, pT7-RpoA::AID, pT7-RpoA::CUT, pT7-RpoA::ppGRN, pT7-RpoA::GAD₄₄₈₋₅₈₅, and pT7-RpoA::Nacht) was referred to as BL21 (DE3):pT7-SlyD (BL21 (DE3):pT7-SlyD::mp-INS, BL21 (DE3):pT7-RpoA::FTN-L, BL21 (DE3):pT7-RpoA::IL-2, BL21 (DE3):pT7-RpoA::FTN-L, BL21 (DE3):pT7-RpoA::IL-2, BL21 (DE3):pT7-RpoA:: EGF, BL21 (DE3):pT7-RpoA::G-CSF, BL21 (DE3):pT7-RpoA::AID, BL21 (DE3):pT7-RpoA::CUT, BL21 (DE3):pT7-RpoA::ppGRN, BL21 (DE3):pT7-RpoA::GAD₄₄₈₋₅₈₅, and BL21 (DE3):pT7-RpoA::Nacht). A portion of the inoculum medium cultured overnight in the LB medium of the colony was inoculated into the LB medium containing 100 μg/ml ampicillin and cultured at 37 and 200 rpm. When the OD₆₀₀ was reached at 0.5, the expression of the recombinant gene was induced by adding 1 mM IPTG. After the addition of IPTG, the medium was cultured under the same conditions for additional 3 to 4 hours.

### <4-1-2> Confirmation of a Soluble Recombinant Protein Produced Using SDS-PAGE

The fungi precipitate was collected by centrifuge of the E. coli cultured by the method in the embodiment <4-1-1> at 6,000 rpm for 5 minutes, suspended in 5 ml of lysed solution (10 mM Tris-HCl buffer, pH 7.5, 10 mM EDTA), and subsequently lysed using an ultrasonic homogenizer (Branson sonifier, USA). After the lysis, the suspension was centrifuged at 13,000 rpm for 10 minutes and as a result, the supernatant and the insoluble inclusion bodies were separated. The protein concentration of the separated supernatant was measured using a Bio-Rad protein assay kit (USA). The supernatant and the insoluble inclusion bodies were mixed with 5×SDS (0.156 M Tris-HCl, pH 6.8, 2.5% SDS, 37.5% glycerol, 37.5 mMDTT) at the ratio of 1:4, respectively, and the mixture was boiled at 100° C. for 10 minutes. The boiled samples were loaded into the wells of 10% SDS-PAGE gel and developed at 125 V for 2 hours. The gel was stained by the Coomassie staining method and then decolorized. The amount of expression of each recombinant protein was confirmed using a densitometer (Bio-Rad, USA) and then the solubility (%) was calculated according to the following formula 1.

$$Solubility ( \% ) = \frac{total amount of supernatant protein ( μ g ) \times 100}{\begin{matrix}total amount of supernatant protein ( μ g ) + \\ total amount of precipitate protein ( μ g )\end{matrix}}$$

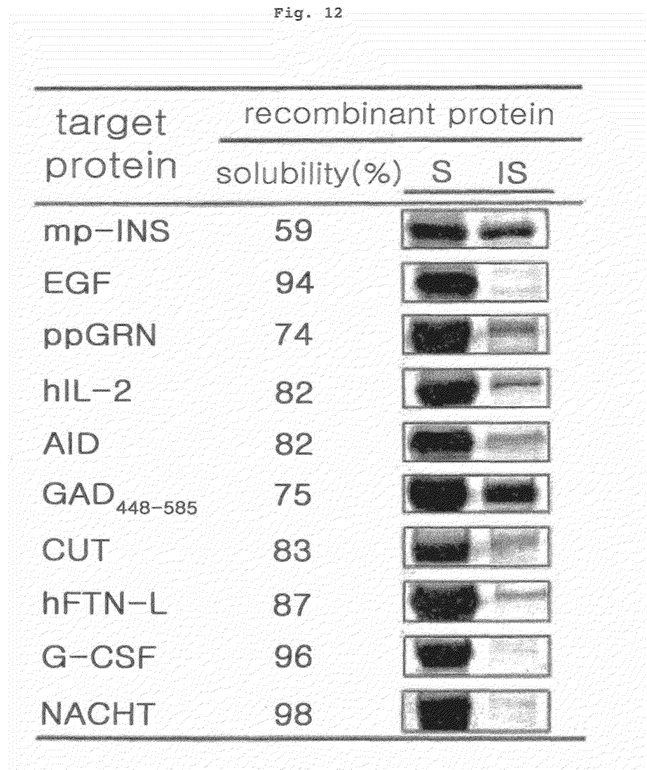

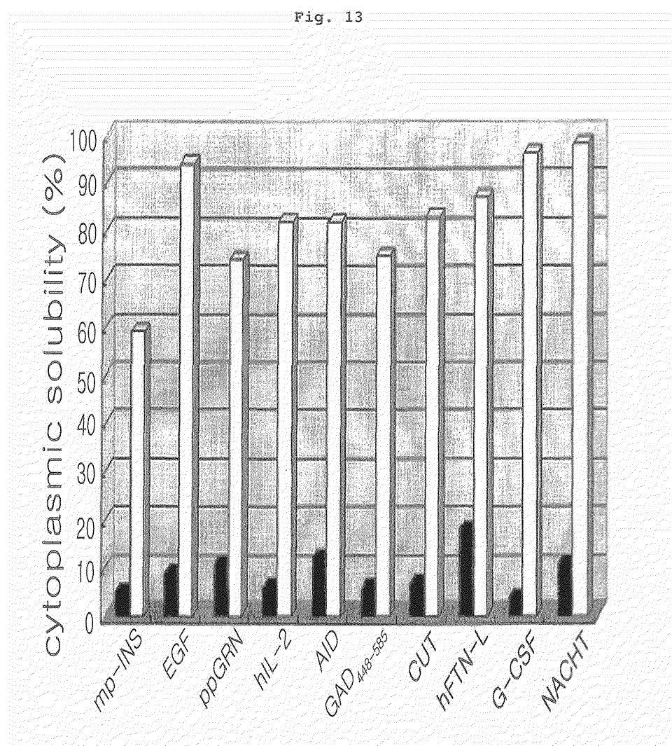

As a result, it was confirmed that the water-soluble expression of the heterologous protein fused with the SlyD was more abundant than those of most insoluble inclusion bodies (FIG. 12). In addition, it was confirmed that the solubility in a heterologous protein fused with the SlyD was increased more than that in a heterologous protein expressed alone (FIG. 13).

<4-1-3> Enzyme Activity of Cutinase Fusion Expressed with the SlyD

A hydrolysis activity of a cutinase fusion expressed with the SlyD (hereinafter, referred to as “SlyD:CUT”) obtained in the embodiments <4-1-1> and <4-1-2> was measured.

After 106.7 μl of 0.1 M phosphate buffer (pH 8.0) and 13.3 μl of Triton X-100 (4 g/L) was placed into a 96-well microplate, each of 13.3 μl of a crude SlyD::CUT supernatant and a supernatant of E. coli BL21 (DE3) as a control group was added into it. A hydrolysis reaction was initiated by adding 66.7 μl of a substrate (PNP (p-nitrophenyl palmitate) and PNB (p-nitrophenyl butyrate), 6.6 mM; Sigma, USA) into the solution, and allowed to react at 37° C. for 20 minutes. After the reaction began, the change in optical density was measured on a Bio-Rad microplate reader (USA) at 415 nm wavelength per minute.

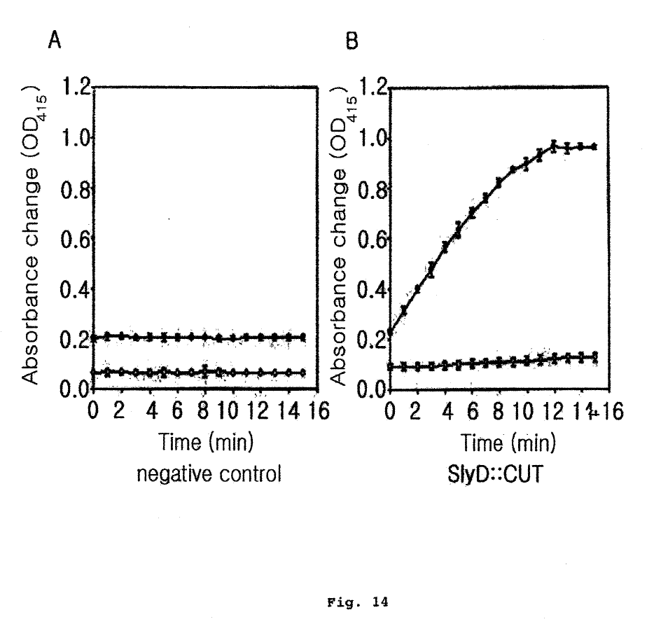

The result showed that the water soluble recombinant protein SlyD::CUT had a PNP-specific degradation ability (FIG. 14).

<4-2> Confirmation of the Water-Soluble Expression of the Heterologous Protein Fused with the Fusion Expression Partner Crr
<4-2-1> Transformation in E. coli and Expression of a Recombinant Protein

The expression vector prepared in the embodiment <3-2-2> was transformed in the E. coli by the same method as in the embodiment <4-1-1>.

### <4-2-2> Confirmation of a Soluble Recombinant Protein Produced Using SDS-PAGE

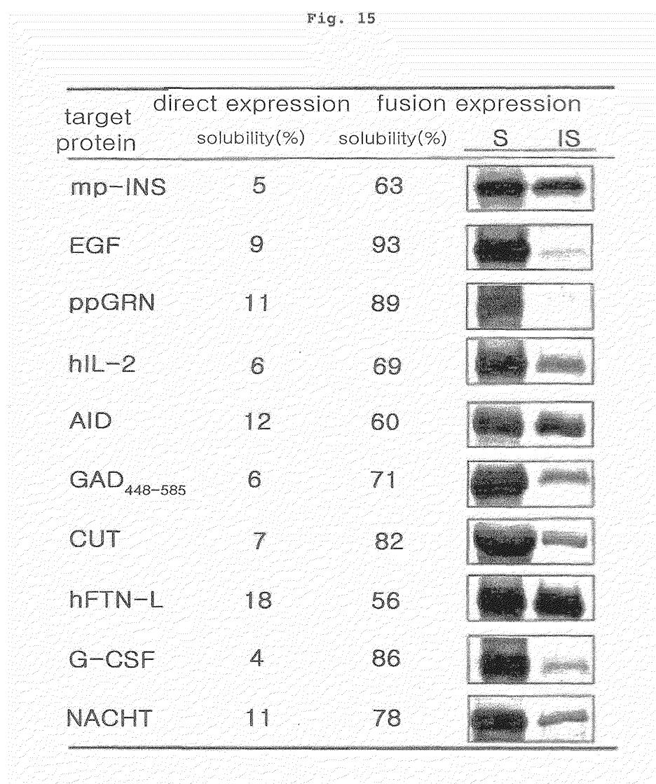

It was confirmed that a protein in the E. coli cultured by the method in the embodiment <4-2-1> was the recombinant protein prepared by the same method as in the embodiment <4-1-2>. As a result, it was confirmed that the water-soluble expression of the heterologous protein fused with the SlyD was more abundant than those of most insoluble inclusion bodies (FIG. 15). In addition, it was confirmed that the solubility in a heterologous protein fused with the SlyD was increased more than that in a heterologous protein expressed alone (FIG. 15).

<4-2-3> Study on Enzyme Activity of Cutinase Fusion Expressed with the Crr

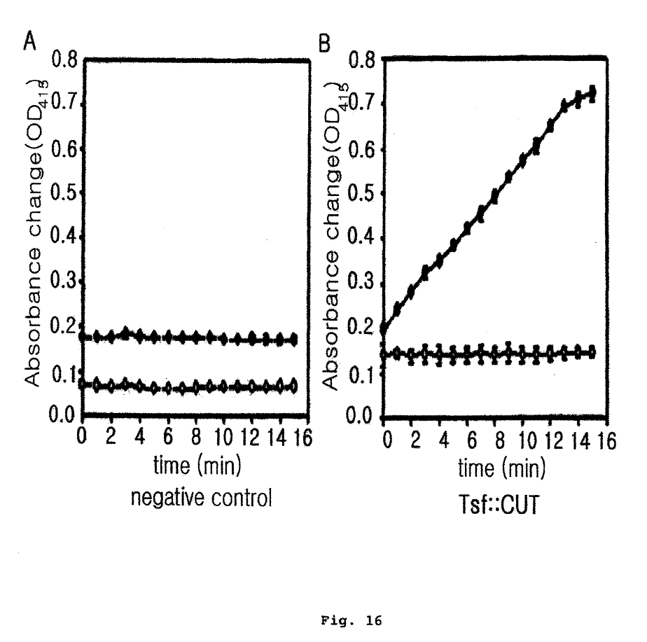

A hydrolysis activity of a cutinase fusion expressed with the Crr (hereinafter, referred to as “Crr:CUT”) obtained in the embodiments <4-2-1> and <4-2-2> was measured. The measurement was carried out by the same method as in the embodiment <4-1-3>. The result showed that the water soluble recombinant protein Crr::CUT had a PNP-specific degradation ability (FIG. 16).

<4-2-4> Confirmation of Conditions of the Heterologous Protein After the Removal of the Crr from the Recombinant Protein

The fusion expression vector containing a recognition site for the protein prepared in the embodiment <3-2-2> was transformed in the E. coliBL21 (DE3) using the method described by Hanahan (Hanahan D, DNA Cloning vol. 1 109-135, IRS press 1985), and the expression of the recombinant gene was induced. The transformed BL21 (DE3) was lysed by the method in the embodiment <5-2>, and then the supernatant and the insoluble inclusion bodies were separated by centrifuge.

A recombinant protein (His₆)Crr::(enterokinase)G-CSF was purified from the supernatant by an affinity chromatography. Specifically, the recombinant protein was combined by adding the supernatant to 500 μl of Ni-NTA agarose beads (Qiagen, USA) and allowed to react overnight at 30° C. by adding 500 μl of an enterokinase solution (5 μl enterokinase, 50 μl 10× buffer, 445 μl PBS; Invitrogen, USA). The eluted fraction was collected and centrifuged at 13,000 rpm for 15 minutes, and the water-soluble supernatant and the insoluble inclusion bodies were separated.

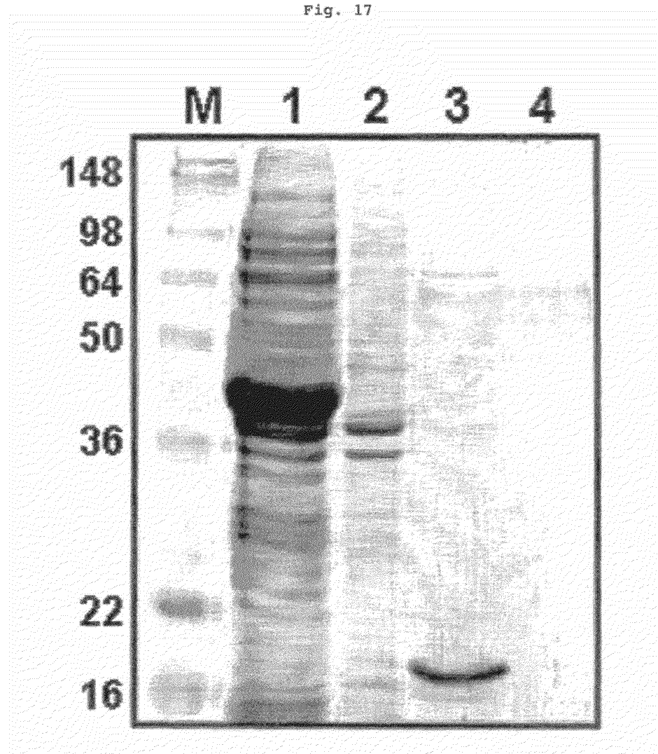

The supernatant and the insoluble inclusion bodies were mixed with 5×SDS (0.156 M Tris-HCl, pH 6.8, 2.5% SDS, 37.5% glycerol, 37.5 mM DTT) at the ratio of 1:4, respectively, and the mixture was boiled at 100° C. for 10 minutes. The boiled samples were loaded into the wells of 10% SDS-PAGE gel and developed at 125 V for 2 hours. The gel was stained by the Coomassie staining method and then decolorized. The amount of expression of each recombinant protein was confirmed. As a result, it was confirmed that the G-CSF with the Crr removed existed in a water-soluble state (FIG. 17).

<4-3> Confirmation of the Water-Soluble Expression of the Heterologous Protein Fused with the Fusion Expression Partner RpoS
<4-3-1> Transformation in E. coli and Expression of a Recombinant Protein

The expression vector prepared in the embodiment <3-2-3> was transformed in E. coli in the same method as in the embodiment <4-1-1>.

### <4-3-2> Confirmation of a Soluble Recombinant Protein Produced Using SDS-PAGE

It was confirmed that a protein in the E. coli cultured by the method in the embodiment <4-3-1> was the recombinant protein prepared by the same method as in the embodiment <4-1-2>.

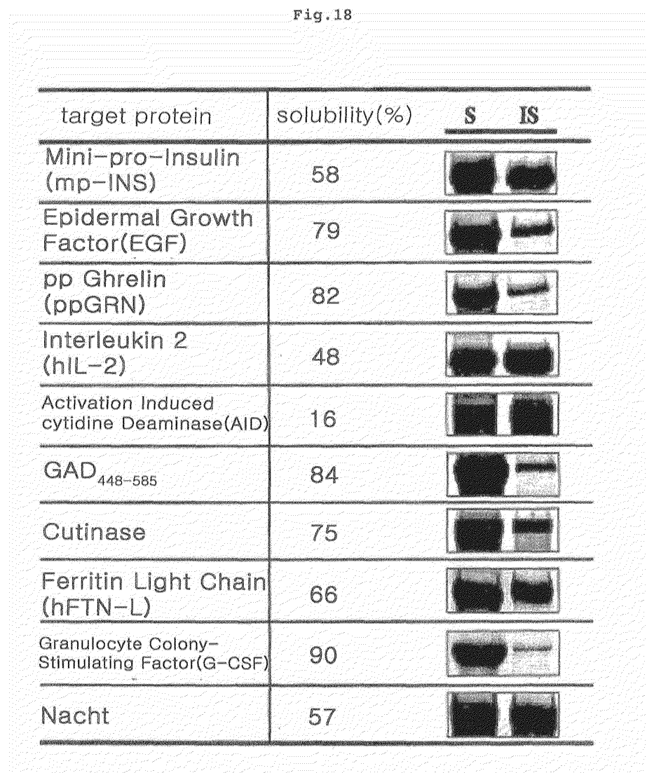

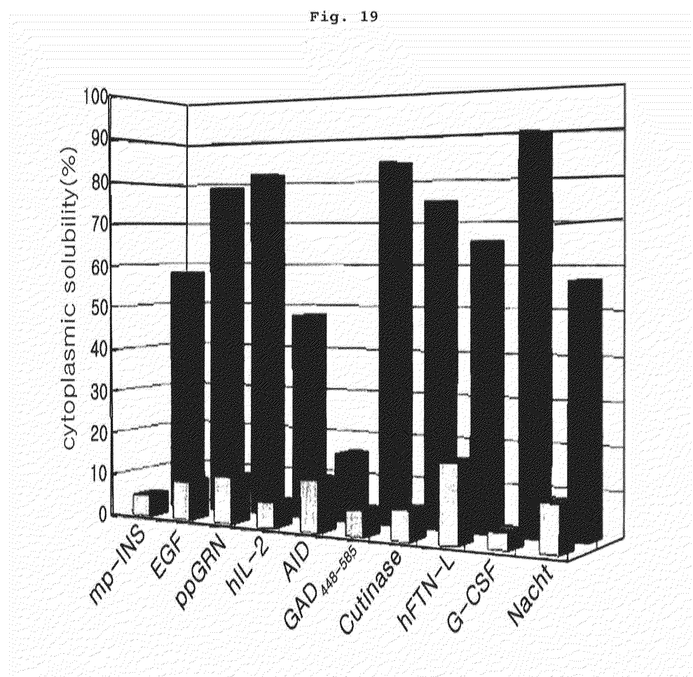

As a result, it was confirmed that the water-soluble expression of the heterologous protein fused with the RpoS was more abundant than those of most insoluble inclusion bodies (FIG. 18). In addition, it was confirmed that the solubility in a heterologous protein fused with the RpoS was increased more than that in a heterologous protein expressed alone (FIG. 19).

<4-3-3> Enzyme Activity of a Cutinase Fusion Expressed with Rpos

A hydrolysis activity of a cutinase fusion expressed with the RpoS (hereinafter, referred to as “RpoS:CUT”) obtained in the embodiments <4-3-1> and <4-3-2> was measured.

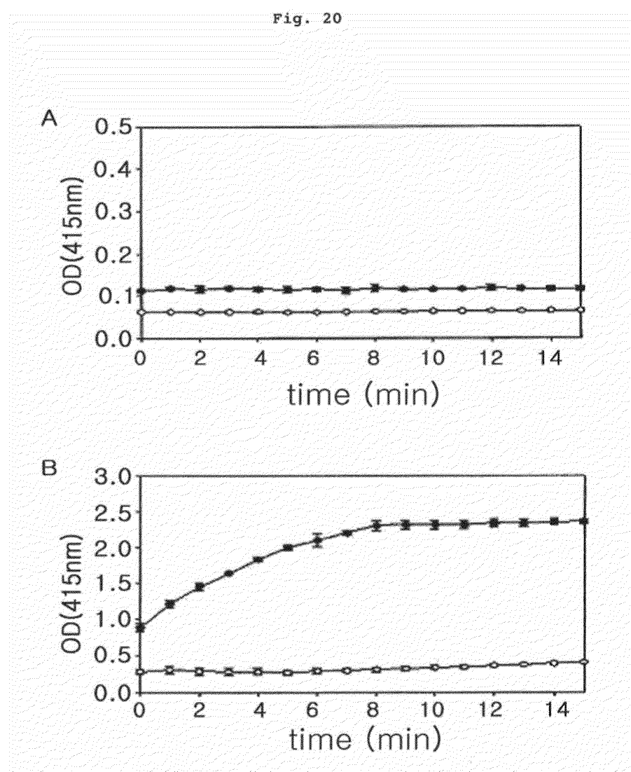

The measurement was carried out by the same method as in the embodiment <4-1-3>. The result showed that the water soluble recombinant protein RpoS::CUT had a PNP-specific degradation ability (FIG. 20).

### <4-3-4> Purification of a Recombinant Protein Using a Histidine Tag

The fusion expression vector for purification containing 6 histidines prepared in the embodiment <3-2-2> was transformed in the E. coliBL21 (DE3) using the method described by Hanahan (Hanahan D, DNA Cloning vol. 1 109-135, IRS press 1985), and the expression of the recombinant gene was induced. The transformed BL21 (DE3) was lysed by the method in the embodiment <5-2>, and then the supernatant and the insoluble inclusion bodies were separated by centrifuge.

A recombinant protein RpoS::CUT-His₆ was purified from the supernatant by an affinity chromatography. Specifically, the ProBond resin (Ni²⁺) column (Invitrogen, USA) filled with metal ions was washed using a binding buffer [pH 8.0, 50 mM sodium phosphate, 300 mM NaCl, 10 mM imidazole] and the supernatant containing the RpoS::CUT-His₆ was combined at 4° C. in the washed column. Subsequently, the mixture was washed twice using 8 μl of a washing buffer (pH 8.0, 50 mM sodium phosphate, 300 mM NaCl, 50 mM imidazole), and the RpoS::CUT-His₆ was collected using an elution buffer (pH 8.0, sodium phosphate, 300 mM NaCl, 250 mM imidazole). To remove the imidazole from the elution buffer containing the RpoS::CUT-His₆, an Amicon Ultra-4 centrifugal filter (Millipore, USA) was used and a buffer exchange was carried out using a PBS buffer (137 mM NaCl, 2.7 mM KCl, 10 mM Na₂HPO₄, 2 mM KH₂PO₄, pH 7.4).

The separated supernatant and the purified RpoS::CUT-His₆ were mixed with 5×SDS (0.156 M Tris-HCl, pH 6.8, 2.5% SDS, 37.5% glycerol, 37.5 mM DTT) at the ratio of 1:4, respectively, and the mixture was boiled at 100° C. for 10 minutes. The boiled samples were loaded into the wells of 10% SDS-PAGE gel and developed at 125 V for 2 hours. The gel was stained by the Coomassie staining method and then decolorized. The amount of expression of each recombinant protein was confirmed.

In addition, the RpoS::CUT-His₆ was analyzed using a HPLC (high-performance liquid chromatography). Specifically, a LC-20A Prominence model (Shimadzu Co. Ltd., Japan) and Shim-pack CLC-NH₂ column (60×150 mm, Shimadzu Co. Ltd., Japan) were used as tools for analysis. Prior to an analysis using the column, an equilibrium was reached by flowing 10% acetonitrile (SIGMA, USA) containing 0.1% trifluoroacetic acids (SIGMA, USA) at 1 ml/min. After the RpoS::CUT-His6 purified by the method was introduced into the equilibrated column, the mixture was eluted from the column by flowing 5% acetonitrile at 1 ml/min. The elution profile was detected using a wavelength at 215 nm.

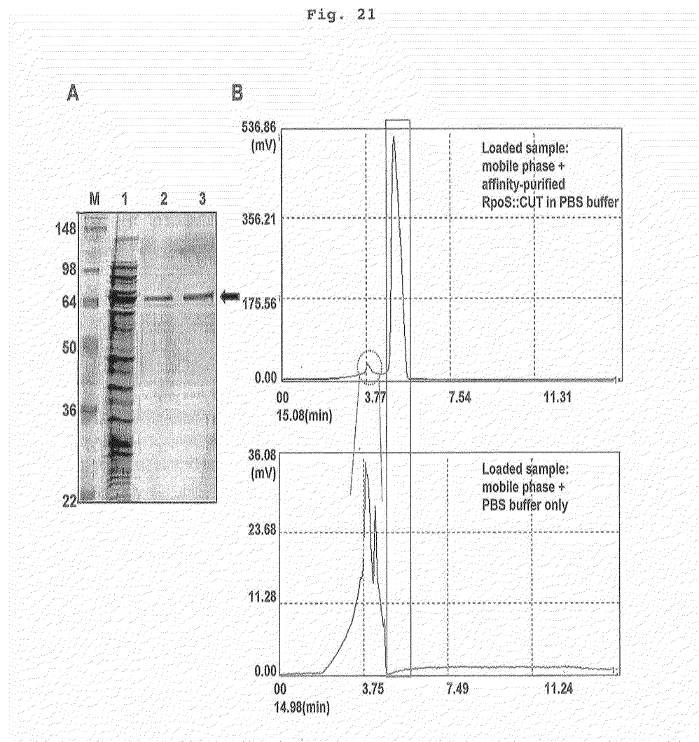

The result showed that only the RpoS::CUT-His6 was purified from the water-soluble supernatant (FIG. 21-A). It was confirmed that there was one peak from the HPLC analysis result (FIG. 21-B).

<4-4> Confirmation of the Water-Soluble Expression of the Heterologous Protein Fused with the Fusion Expression Partner PotD
<4-4-1> Transformation in E. coli and Expression of a Recombinant Protein

The expression vector prepared in the embodiment <3-2-4> was transformed in E. coli in the same method as in the embodiment <4-1-1>.

### <4-4-2> Confirmation of a Soluble Recombinant Protein Produced Using SDS-PAGE

It was confirmed that a protein in the E. coli cultured by the method in the embodiment <4-4-1> was the recombinant protein prepared by the same method as in the embodiment <4-1-2>.

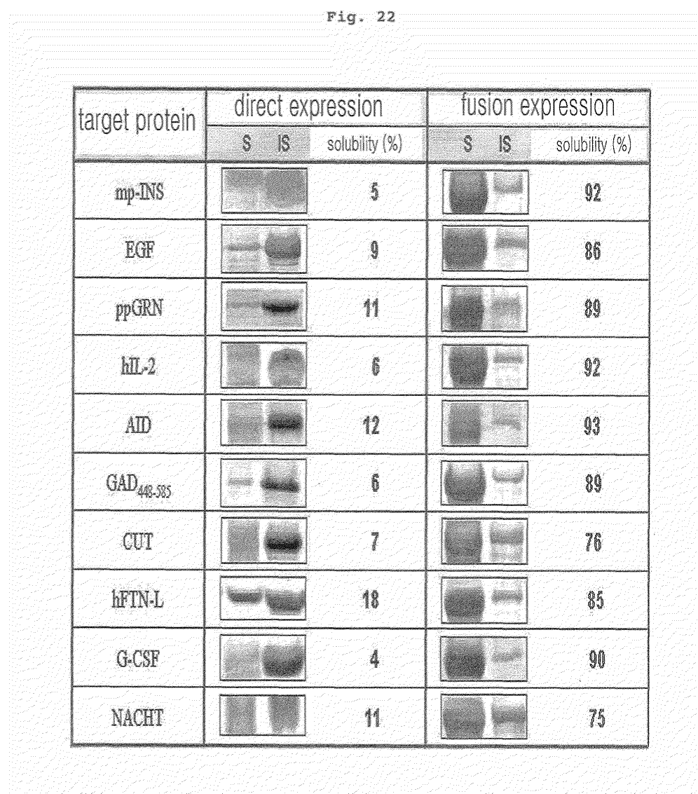

As a result, it was confirmed that the water-soluble expression of the heterologous protein fused with the PotD was more abundant than those of most insoluble inclusion bodies (FIG. 22). In addition, it was confirmed that the solubility in a heterologous protein fused with the PotD was increased more than that in a heterologous protein expressed alone (FIG. 22).

<4-4-3> Enzyme Activity of a Cutinase Fusion Expressed with the PotD

A hydrolysis activity of a cutinase fusion expressed with the PotD (hereinafter, referred to as “PotD:CUT”) obtained in the embodiments <4-4-1> and <4-4-2> was measured.

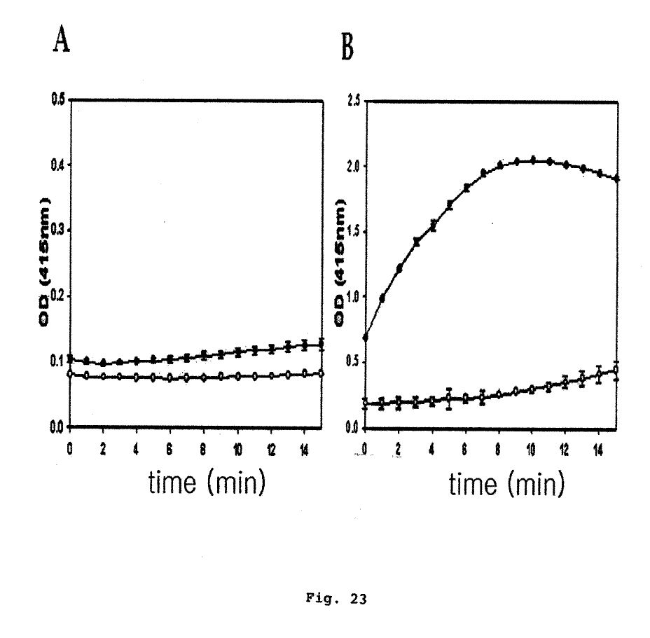

The measurement was carried out by the same method as in the embodiment <4-1-3>. The result showed that the water soluble recombinant protein PotD::CUT had a PNP-specific degradation ability (FIG. 23).

<4-4-4> Removal of the PotD from the Recombinant Protein

The fusion expression vector containing a recognition site for the protein prepared by the method in the embodiment <3-2-4> was transformed in the E. coliBL21 (DE3) using the method described by Hanahan (Hanahan D, DNA Cloning vol. 1 109-135, IRS press 1985), and the expression of the recombinant gene was induced. The transformed BL21 (DE3) was lysed by the method in the embodiment <5-2>, and then the supernatant and the insoluble inclusion bodies were separated by centrifuge.

Subsequently, the experiment was carried out in the same method as in the embodiment <4-2-4>

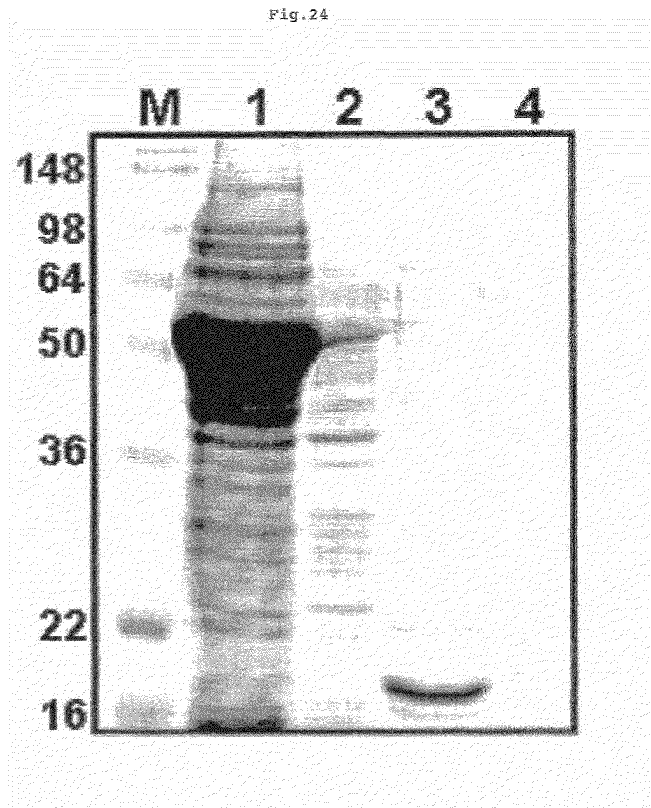

As a result, it was confirmed that the G-CSF with the PotD removed existed in a water-soluble state (FIG. 24).

<4-5> Confirmation of the Water-Soluble Expression of the Heterologous Protein Fused with the Fusion Expression Partner RpoA
<4-5-1> Transformation in E. coli and Expression of a Recombinant Protein

The expression vector prepared in the embodiment <3-2-5> was transformed in E. coli in the same method as in the embodiment <4-1-1>.

### <4-5-2> Confirmation of a Soluble Recombinant Protein Produced Using SDS-PAGE

It was confirmed that a protein in the E. coli cultured by the method in the embodiment <4-5-1> was the recombinant protein prepared by the same method as in the embodiment <4-1-2>.

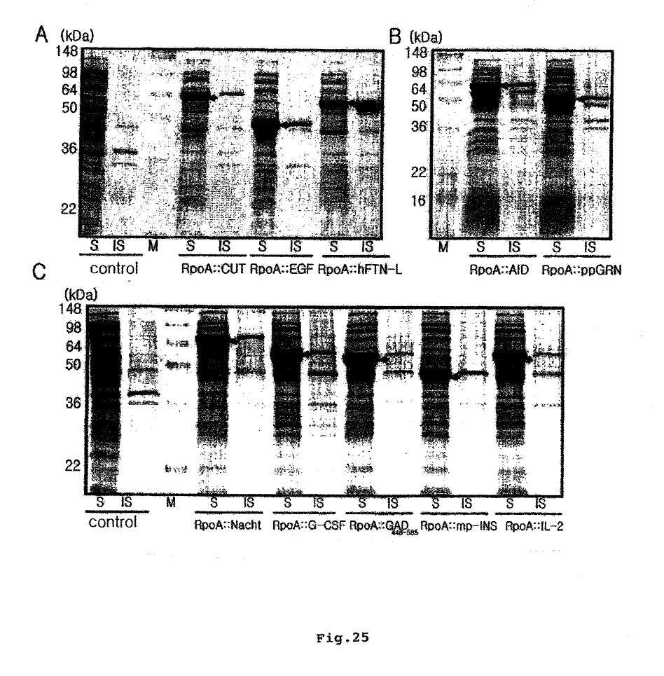

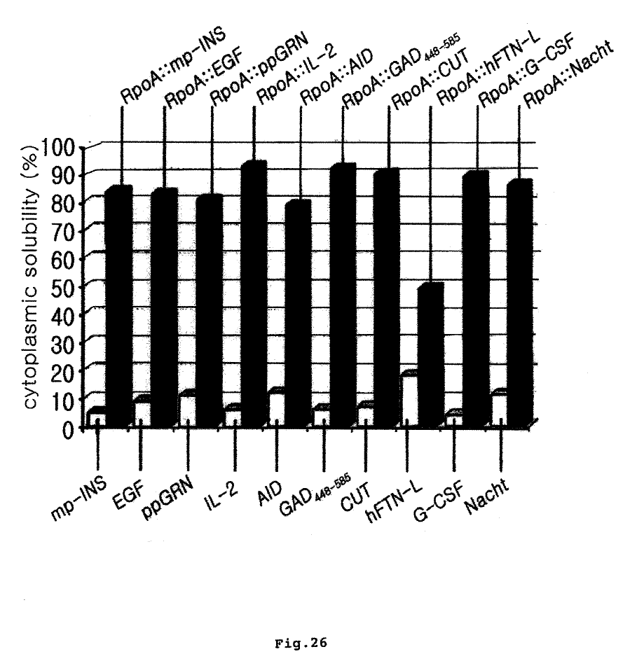

As a result, it was confirmed that the water-soluble expression of the heterologous protein fused with the RpoA was more abundant than those of most insoluble inclusion bodies (FIG. 25). In addition, it was confirmed that the solubility in a heterologous protein fused with the RpoA was increased more than that in a heterologous protein expressed alone (FIG. 26).

<4-5-3> Enzyme Activity of a Cutinase Fusion Expressed with the RpoA

A hydrolysis activity of a cutinase fusion expressed with the PotD (hereinafter, referred to as “RpoA:CUT”) obtained in the embodiments <4-5-1> and <4-5-2> was measured.

The measurement was carried out by the same method as in the embodiment <4-1-3>.

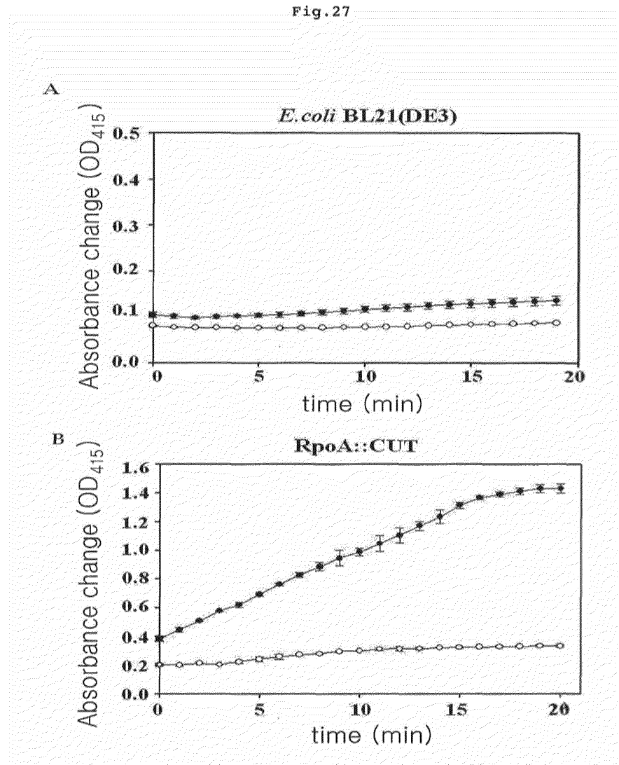

The result showed that the water soluble recombinant protein RpoA::CUT had a PNP-specific degradation ability (FIG. 27).

### INDUSTRIAL APPLICABILITY

The method of producing a recombinant protein using a fusion expression partner selected from the group consisting of SlyD, Crr, RpoS, PotD, and RpoA of the present invention may overcome the limitations about the water-solubility and folding which the conventional fusion expression partners have, and be used widely in the production of pharmaceutical and industrial proteins.

1. A method of preparing a recombinant protein, comprising:
1) preparing an expression vector linking a polynucleotide with a polyDNA fragment encoding a target protein, wherein the polynucleotide encoding a fusion expression partner is Rpos (RNApolymerase sigma factor);
2) preparing a transformant by introducing the expression vector into a host cell; and
3) inducing and obtaining an expression of a recombinant protein by culturing the transformant.

2. The method as set forth in claim 1, wherein the RpoS is represented by SEQ ID NO 1.

3. The method as set forth claim 1, wherein the heterologous protein in step 1) has a biological activity of a protein selected from the group consisting of an antigen, an antibody, a cell receptor, an enzyme, a structural protein, a serum, and a cell protein.

4. An expression vector for production of a recombinant protein, comprising:
a polynucleotide encoding a fusion expression partner and a polyDNA fragment encoding a target protein, wherein the fusion expression partner is Rpos (RNA polymerase sigma factor).

5. The expression vector as set forth in claim 4, wherein the polynucleotide encoding the fusion expression partner is linked with a polynucleotide encoding the heterologous protein.

6. The expression vector as set forth in claim 4, wherein the polynucleotide encoding a protein restriction enzyme recognition site is linked between the polynucleotide encoding the fusion expression partner and the polynucleotide encoding the heterologous protein.

7. A transformant transduced with the expression vector of claim 4.

8. (canceled)


SEQUENCE LISTING (60 sequences)

SEQ ID NO: 1 - DNA, 591 bp, Escherichia coli
ttagtggcaaccgcaaccgccgttgcctttaccgccacagcagccttcgccaccgtgttcatgaccgtgatcgtggccatgaccgccgcagcaaccgtcgtggtcgtgatcgtggtggtgatcgtgcgcgccgtgaacgtgaccatgagccagttcttcttcagtcgcttcgcgaatcgccacaacttcaacgttgaatttcaggttctgaccggccagcatgtggttaccatcaaccacgacgtgatcgtcttcaaccgcagtgatttcaaccggtaccggaccctggtcggtttcagccaggaaacgcatacctacctgcagttcatcaacgcccataaatacgtctttaggaacacgttgcaccaggttttcgtcgtactgaccgtaagcgtcgttcgcgccaacagcgacatcaaatttgtcgccaacttcatgaccttccagcgccgtttccaggccagagatcagggaaccgtgaccatgcaggtagtccagcggcgcactcaccggagactcatcaaccaacacaccgtcttctgtacgtacctgataggccaggctgaccaccaggtcttttgctactttcat

SEQ ID NO: 2 - DNA, 24 bp, Artificial Sequence
  SlyD sense primer
catatgaaagtagcaaaagacctg

SEQ ID NO: 3 - DNA, 27 bp, Artificial Sequence
  SlyD antisense primer
ctcgaggtggcaaccgcaaccgccgtt

SEQ ID NO: 4 - DNA, 510 bp, Escherichia coli
atgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgtccggtagcgtaaccgtgggtgaaaccccggttatccgcatcaagaagtaa

SEQ ID NO: 5 - DNA, 24 bp, Artificial Sequence
  Crr sense primer
catatgggtttgttcgataaactg

SEQ ID NO: 6 - DNA, 24 bp, Artificial Sequence
  Crr antisense primer
ctcgagcttcttgatgcggataac

SEQ ID NO: 7 - DNA, 993 bp, Escherichia coli
ttactcgcggaacagcgcttcgatattcagcccctgcgtttgcaggatttcgcgcaaacggcgcaggccttcaacctgaatctggcgaacacgttcacgggtgaggccaatttcacgacctacatcttccagtgttgccgcttcgtaccccagcaaaccgaatcgacgtgccagcacttcacgctgtttggcgttcagctcgaacagccatttgacgatgctctgcttcatatcgtcatcttgcgtggtatcttccggaccgttctctttttcatcggccaggatgtccagcaacgctttttcggaatcaccacccagcggggtgtctaccgaggtaatgcgctcgttaagacgaagcatacggctgacgtcatcaactggcttatccagttgctctgcgatctcttccgcacttggttcatggtccagcttatgggacaactcacgtgcggttcgcaggtaaacgttcagctcctttacgatgtgaatcggcaaacgaatagtacgggtttggttcataatcgcccgttcaatcgtctggcgaatccaccaggttgcgtatgttgagaagcggaaaccacgttccgggtcaaacttctctaccgcgcggatcagccccaggttgccctcttcgataaggtccagcaacgccagaccacgattgccataacggcgggcaatttttaccaccagacgcaagttactctcgatcatccggcggcgagaggcgacatctccacgcagtgcgcgacgcgcaaaataaacttcttcttcggccgttaacagtggtgaataaccaatctcaccaaggtaaagctgagtcgcgtccaacacacgctgtgtggctccctgcgataacagttcctcttcggccaaatcgttatcactgggttcctgttctactaaggccttttcgtcaaaaacctcaactccgttctcatcaaattccgcatcttcatttaaatcatgaactttcagcgtattctgactcat

SEQ ID NO: 8 - DNA, 24 bp, Artificial Sequence
  RpoS sense primer
catatgagtcagaatacgctgaaa

SEQ ID NO: 9 - DNA, 24 bp, Artificial Sequence
  RpoS antisense primer
ctcgagctcgcggaacagcgcttc

SEQ ID NO: 10 - DNA, 1047 bp, Escherichia coli
ttaacgtcctgctttcagcttctgataatactcttcataaatgctgctggctgcgccaacgtcattctgccattcgccatttttaatggtttcagcatccgggtagagtgttttatcgttcgccacttctggacttaacagcttacgcgccgcaaggtttggcgttggataaccgatagtttcagcaacctgttttgccacatccgggcgcagcaggaagttgatcaatttcagcgcgccttctttgtttttggcatttgccgggatcgccaggctgtccatccagaaaatgccgccttctttcggccacaccacgtcaattggcgtacccgcctggcgtgcaacgaaagcagaaccgttccagatcatgccgaggttaacttcgccttccatgtacgggttcgccgggttatcggagttaaaggctgccacgtttggcatcagttttttcagctcgttatatgcagcttcaatctctttcggatcggtggtgttaccggagtagcccagcttacgcagcgccatctggaacacttcacgggcatcgtcggtcaacagcaggctgcctttgtactctggcttccacagatcggcccagctggtgacagatttcggatccaccgcatcaccgttaacaccaatcgccgtcgcaccccagatatacggaatggagtagtcgttattcgggtcaaaaggcttgttgagcatgtctggatcgagattgctgaagtttgttaacttcgacttgtcgatcttctggatcatcccttctttacgcattttatcaacgtaataggttgaaggaaccaccagatcataggcaccgtctttgtatgttttcagcttggcgtacatggtttcgttcgactcgtaagtcgaatagataaccttaataccggtttctttggtgaactgttcaagcagtcctggcggcacgtactcggtccagttgtagaaatacagcgtgttgttgtcatcggcgtgagcggcgctcatgcccagtgccagagcacccgccgcgagcaggtggcgtgaccattttttcat

SEQ ID NO: 11 - DNA, 24 bp, Artificial Sequence
  PotD sense primer
catatgaaagtcgcagtcctcggc

SEQ ID NO: 12 - DNA, 24 bp, Artificial Sequence
  PotD antisense primer
ctcgagacgtcctgctttcagctt

SEQ ID NO: 13 - DNA, 990 bp, Escherichia coli
atgcagggttctgtgacagagtttctaaaaccgcgcctggttgatatcgagcaagtgagttcgacgcacgccaaggtgacccttgagcctttagagcgtggctttggccatactctgggtaacgcactgcgccgtattctgctctcatcgatgccgggttgcgcggtgaccgaggttgagattgatggtgtactacatgagtacagcaccaaagaaggcgttcaggaagatatcctggaaatcctgctcaacctgaaagggctggcggtgagagttcagggcaaagatgaagttattcttaccttgaataaatctggcattggccctgtgactgcagccgatatcacccacgacggtgatgtcgaaatcgtcaagccgcagcacgtgatctgccacctgaccgatgagaacgcgtctattagcatgcgtatcaaagttcagcgcggtcgtggttatgtgccggcttctacccgaattcattcggaagaagatgagcgcccaatcggccgtctgctggtcgacgcatgctacagccctgtggagcgtattgcctacaatgttgaagcagcgcgtgtagaacagcgtaccgacctggacaagctggtcatcgaaatggaaaccaacggcacaatcgatcctgaagaggcgattcgtcgtgcggcaaccattctggctgaacaactggaagctttcgttgacttacgtgatgtacgtcagcctgaagtgaaagaagagaaaccagagttcgatccgatcctgctgcgccctgttgacgatctggaattgactgtccgctctgctaactgccttaaagcagaagctatccactatatcggtgatctggtacagcgtaccgaggttgagctccttaaaacgcctaaccttggtaaaaaatctcttactgagattaaagacgtgctggcttcccgtggactgtctctgggcatgcgcctggaaaactggccaccggcaagcatcgctgacgagtaa

SEQ ID NO: 14 - DNA, 24 bp, Artificial Sequence
  RpoA sense primer
catatgcagggttctgtgacagag

SEQ ID NO: 15 - DNA, 27 bp, Artificial Sequence
  RpoA antisense primer
ctcgagttactcgtcagcgatgcttgc

SEQ ID NO: 16 - DNA, 180 bp, Artificial Sequence
  human minipro-insulin
tttgtcaaccaacatttatgtggatcacatttagtagaggctttgtatcttgtttgtggtgaacgtggatttttctatacacctaagacacgtagatctcctaatggaaaacgtggtattgttgaacaatgctgtacatcaatctgttcattgtatcaacttgagaactactgtaactaa

SEQ ID NO: 17 - DNA, 948 bp, Artificial Sequence
  human GAD448-585
accgtggtgttccagggggcggcagggattgggaaaacaatcctggcccggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcataaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctg

SEQ ID NO: 18 - DNA, 780 bp, Artificial Sequence
  Pseudomonas putida cutinase
atggctcccctgccggatacaccgggagcgccatttccggctgtcgccaatttcgaccgcagtggcccctacaccaccagcagccagagcgaggggccgagctgtcgcatctatcggccccgcgacctgggtcaggggggcgtgcgtcatccggtgattctctggggcaatggcaccggtgccgggccgtccacctatgccggcttgctatcgcactgggcaagccacggtttcgtggtggcggcggcggaaacctccaatgccggtaccgggcgggaaatgctcgcctgcctggactatctggtacgtgagaacgacaccccctacggcacctattccggcaagctcaataccgggcgagtcggcacttctgggcattcccagggtggtggcggctcgatcatggccgggcaggatacgagggtgcgtaccacggcgccgatccagccctacaccctcggcctggggcacgacagcgcctcgcagcggcggcagcaggggccgatgttcctgatgtccggtggcggtgacaccatcgcctttccctacctcaacgctcagccggtctaccggcgtgccaatgtgccggtgttctggggcgaacggcgttacgtcagccacttcgagccggtcggtagcggtggggcctatcgcggcccgagcacggcatggttccgcttccagctgatggatgaccaagacgcccgcgctaccttctacggcgcgcagtgcagtctgtgcaccagcctgctgtggtcggtcgagcgccgcgggctttaa

SEQ ID NO: 19 - DNA, 528 bp, Artificial Sequence
  human ferritin light chain
atgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgcgctctcttccaggacatcaagaagccagctgaagatgagtggggtaaaaccccagacgccatgaaagctgccatggccctggagaaaaagctgaaccaggcccttttggatcttcatgccctgggttctgcccgcacggacccccatctctgtgacttcctggagactcacttcctagatgaggaagtgaagctcatcaagaagatgggtgaccacctgaccaacctccacaggctgggtggcccggaggctgggctgggcgagtatctcttcgaaaggctcactctcaagcacgactaa

SEQ ID NO: 20 - DNA, 414 bp, Artificial Sequence
  nacht domain of cryopyrin
cgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagattta

SEQ ID NO: 21 - DNA, 21 bp, Artificial Sequence
  mp-INS sense primer
catatgtttgtcaaccaacat

SEQ ID NO: 22 - DNA, 22 bp, Artificial Sequence
  mp-INS antisense primer
aagcttttagttacagtagttc

SEQ ID NO: 23 - DNA, 24 bp, Artificial Sequence
  EGF sense primer
catatgaactctgactccgaatgc

SEQ ID NO: 24 - DNA, 24 bp, Artificial Sequence
  EGF antisense primer
aagcttttaacgcagttcccacca

SEQ ID NO: 25 - DNA, 21 bp, Artificial Sequence
  ppGRN sense primer
catatgggctccagcttcctg

SEQ ID NO: 26 - DNA, 19 bp, Artificial Sequence
  ppGRN antisense primer
aagctttcacttgtcggct

SEQ ID NO: 27 - DNA, 21 bp, Artificial Sequence
  hIL-2 sense primer
catatggcacctacttcaagt

SEQ ID NO: 28 - DNA, 21 bp, Artificial Sequence
  hIL-2 antisense primer
aagcttttatcaagtcagtgt

SEQ ID NO: 29 - DNA, 24 bp, Artificial Sequence
  AID sense primer
catatggacagcctcttgatgaac

SEQ ID NO: 30 - DNA, 23 bp, Artificial Sequence
  AID antisense primer
aagctttcataacaaaagtccca

SEQ ID NO: 31 - DNA, 20 bp, Artificial Sequence
  GAD448-585 sense primer
catatgcgccacgttgatgt

SEQ ID NO: 32 - DNA, 21 bp, Artificial Sequence
  GAD448-585 antisense primer
atcgatttataaatcttgtcc

SEQ ID NO: 33 - DNA, 23 bp, Artificial Sequence
  CUT sense primer
catatggctcccctgccggatac

SEQ ID NO: 34 - DNA, 23 bp, Artificial Sequence
  CUT antisense primer
aagcttttaaagcccgcggcgct

SEQ ID NO: 35 - DNA, 21 bp, Artificial Sequence
  hFTN-L sense primer
catatgagctcccagattcgt

SEQ ID NO: 36 - DNA, 24 bp, Artificial Sequence
  hFTN-L antisense primer
aagcttttagtcgtgcttgagagt

SEQ ID NO: 37 - DNA, 22 bp, Artificial Sequence
  G-CSF sense primer
catatgactccactcggacctg

SEQ ID NO: 38 - DNA, 21 bp, Artificial Sequence
  G-CSF antisense primer
aagctttcatggctgtgcaag

SEQ ID NO: 39 - DNA, 21 bp, Artificial Sequence
  Nacht sense primer
catatgactgtggtgttccag

SEQ ID NO: 40 - DNA, 22 bp, Artificial Sequence
  Nacht antisense primer
aagctttcacagcaggtagtac

SEQ ID NO: 41 - DNA, 21 bp, Artificial Sequence
  mp-INS sense primer
ctcgagtttgtcaaccaacat

SEQ ID NO: 42 - DNA, 22 bp, Artificial Sequence
  mp-INS antisense primer
aagcttttagttacagtagttc

SEQ ID NO: 43 - DNA, 24 bp, Artificial Sequence
  EGF sense primer
ctcgagaactctgactccgaatgc

SEQ ID NO: 44 - DNA, 24 bp, Artificial Sequence
  EGF antisense primer
aagcttttaacgcagttcccacca

SEQ ID NO: 45 - DNA, 21 bp, Artificial Sequence
  ppGRN sense primer
ctcgagggctccagcttcctg

SEQ ID NO: 46 - DNA, 19 bp, Artificial Sequence
  ppGRN antisense primer
aagctttcacttgtcggct

SEQ ID NO: 47 - DNA, 21 bp, Artificial Sequence
  hIL-2 sense primer
ctcgaggcacctacttcaagt

SEQ ID NO: 48 - DNA, 21 bp, Artificial Sequence
  hIL-2 antisense primer
aagcttttatcaagtcagtgt

SEQ ID NO: 49 - DNA, 21 bp, Artificial Sequence
  GAD448-585 antisense primer
atcgatttataaatcttgtcc

SEQ ID NO: 50 - DNA, 24 bp, Artificial Sequence
  AID sense primer
ctcgaggacagcctcttgatgaac

SEQ ID NO: 51 - DNA, 23 bp, Artificial Sequence
  AID antisense primer
aagctttcataacaaaagtccca

SEQ ID NO: 52 - DNA, 20 bp, Artificial Sequence
  GAD448-585 sense primer
ctcgagcgccacgttgatgt

SEQ ID NO: 53 - DNA, 23 bp, Artificial Sequence
  CUT sense primer
ctcgaggctcccctgccggatac

SEQ ID NO: 54 - DNA, 23 bp, Artificial Sequence
  CUT antisense primer
aagcttttaaagcccgcggcgct

SEQ ID NO: 55 - DNA, 21 bp, Artificial Sequence
  hFTN-L sense primer
ctcgagagctcccagattcgt

SEQ ID NO: 56 - DNA, 24 bp, Artificial Sequence
  hFTN-L antisense primer
aagcttttagtcgtgcttgagagt

SEQ ID NO: 57 - DNA, 22 bp, Artificial Sequence
  G-CSF sense primer
ctcgagactccactcggacctg

SEQ ID NO: 58 - DNA, 21 bp, Artificial Sequence
  G-CSF antisense primer
aagctttcatggctgtgcaag

SEQ ID NO: 59 - DNA, 21 bp, Artificial Sequence
  Nacht sense primer
ctcgagactgtggtgttccag

SEQ ID NO: 60 - DNA, 22 bp, Artificial Sequence
  Nacht antisense primer
aagctttcacagcaggtagtac